SEQUENCE LISTING (4 sequences)

SEQ ID NO: 1 - DNA, 4432 bp, Homo sapiens
  CDS (16)..(4416)
gctagcgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagctcgaggaattcgtcgacggtaccgggcccatgtttctgtacaacttaaccttgcagagagccactggcatcagctttgccattcatggaaacttttctggaaccaaacaacaagaaattgttgtttcccgtgggaagatcttggagctgcttcgcccagaccccaacactggcaaagtacataccctactcactgtggaagtattcggtgttatccggtcactcatggcctttaggctgacaggtggcaccaaagactacattgtagttggcagtgactctggtcgaattgttattttggaataccagccatctaagaatatgtttgagaagattcaccaagaaacctttggcaagagtggatgccgtcgcatcgttcctggccagttcttagctgtggatcccaaagggcgagccgttatgattagtgccattgagaaacagaaattggtgtatattttgaacagagatgctgcagcccgacttaccatttcatctcccctggaagcccacaaagcaaacactttagtgtatcatgtagttggagtagatgtcggatttgaaaatccaatgtttgcttgtctggaaatggattatgaggaagcagacaatgatccaacaggggaagcagcagctaatacccagcagacacttactttctatgagctagaccttggtttaaatcatgtggtccgaaaatacagtgaacctttggaggaacacggcaacttccttattacagttccaggagggtcagatggtccaagtggagtactgatctgctctgaaaactatattacttacaagaactttggtgaccagccagatatccgctgtccaattcccaggaggcggaatgacctggatgaccctgaaagaggaatgatttttgtctgctctgcaacccataaaaccaaatcgatgttcttctttttggctcaaactgagcagggagatatctttaagatcactttggagacagatgaagatatggttactgagatccggctcaaatattttgatactgtacccgttgctgctgccatgtgtgtgcttaaaacagggttcctttttgtagcatcagaatttggaaaccattacttatatcaaattgcacatcttggagatgatgatgaagaacctgagttttcatcagccatgcctctggaagaaggagacacattcttttttcagccaagaccacttaaaaaccttgtgctggttgatgagttggacagcctctctcccattctgttttgccagatagctgatctggccaatgaagatactccacagttgtatgtggcctgtggtaggggaccccgatcatctctgagagtcctaagacatggacttgaggtgtcagaaatggctgtttctgagctacctggtaaccccaacgctgtctggacagtgcgtcgacacattgaagatgagtttgatgcctacatcattgtgtctttcgtgaatgccaccctagtgttgtccattggagaaactgtagaagaagtgactgactctgggttcctggggaccaccccgaccttgtcctgctccttattaggagatgatgccttggtgcaggtctatccagatggcattcggcacatacgagcagacaagagagtcaatgagtggaagacccctggaaagaaaacaattgtgaagtgtgcagtgaaccagcgacaagtggtgattgccctgacaggaggagagctggtctatttcgagatggatccttcaggacagctgaatgagtacacagaacggaaggagatgtcagcagatgtggtgtgcatgagtctggccaatgtaccccctggagagcagcggtctcgcttcctggctgtggggcttgtggacaacactgtcagaatcatctccctggatccctcagactgtttgcaacctctaagcatgcaggctctcccagcccagcctgagtccttgtgtatcgtggaaatgggtgggactgagaagcaggatgagctgggtgagaggggctcgattggcttcctatacctgaatattgggctacagaacggtgtgctgctgaggactgtcttggaccctgtcactggggatttgtctgatactcgcactcggtacctggggtcccgtcctgtgaagctcttccgagtccgaatgcaaggccaggaggcagtattggccatgtcaagccgctcatggttgagctattcttaccaatctcgcttccatctcaccccactgtcttacgagacactggaatttgcatcgggttttgcctcggaacagtgtcccgagggcattgtggccatctccaccaacaccctacggattttggcattagagaagctcggtgctgtcttcaatcaagtagccttcccactgcagtacacacccaggaaatttgtcatccaccctgagagtaacaaccttattatcattgaaacggaccacaatgcctacactgaggccacgaaagctcagagaaagcagcagatggcagaggaaatggtggaagcagcaggggaggatgagcgggagctggccgcagagatggcagcagcattcctcaatgaaaacctccctgaatccatctttggagctcccaaggctggcaatgggcagtgggcctctgtgatccgagtgatgaatcccattcaagggaacacactggaccttgtccagctggaacagaatgaggcagcttttagtgtggctgtgtgcaggttttccaacactggtgaagactggtatgtgctggtgggtgtggccaaggacctgatactaaacccccgatctgtggcagggggcttcgtctatacttacaagcttgtgaacaatggggaaaaactggagtttttgcacaagactcctgtggaagaggtccctgctgctattgccccattccaggggagggtgttgattggtgtggggaagctgttgcgtgtctatgacctgggaaagaagaagttactccgaaaatgtgagaataagcatattgccaattatatctctgggatccagactatcggacatagggtaattgtatctgatgtccaagaaagtttcatctgggttcgctacaagcgtaatgaaaaccagcttatcatctttgctgatgatacctacccccgatgggtcactacagccagcctcctggactatgacactgtggctggggcagacaagtttggcaacatatgtgtggtgaggctcccacctaacaccaatgatgaagtagatgaggatcctacaggaaacaaagccctgtgggaccgtggcttgctcaatggggcctcccagaaggcagaggtgatcatgaactaccatgtcggggagacggtgctgtccttgcagaagaccacgctgatccctggaggctcagaatcacttgtctataccaccttgtctggaggaattggcatccttgtgccattcacgtcccatgaggaccatgacttcttccagcatgtggaaatgcacctgcggtctgaacatccccctctctgtgggcgggaccacctcagctttcgctcctactacttccctgtgaagaatgtgattgatggagacctctgtgagcagttcaattccatggaacccaacaaacaaaagaacgtctctgaagaactggaccgaaccccacccgaagtgtccaagaaactcgaggatatccggacccgctacgccttctgaaagcttaagtttaaac

SEQ ID NO: 2 - protein, 1466 aa, Homo sapiens
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLEEFVDGTGPMFLYNLTLQRATGISFAIHGNFSGTKQQEIVVSRGKILELLRPDPNTGKVHTLLTVEVFGVIRSLMAFRLTGGTKDYIVVGSDSGRIVILEYQPSKNMFEKIHQETFGKSGCRRIVPGQFLAVDPKGRAVMISAIEKQKLVYILNRDAAARLTISSPLEAHKANTLVYHVVGVDVGFENPMFACLEMDYEEADNDPTGEAAANTQQTLTFYELDLGLNHVVRKYSEPLEEHGNFLITVPGGSDGPSGVLICSENYITYKNFGDQPDIRCPIPRRRNDLDDPERGMIFVCSATHKTKSMFFFLAQTEQGDIFKITLETDEDMVTEIRLKYFDTVPVAAAMCVLKTGFLFVASEFGNHYLYQIAHLGDDDEEPEFSSAMPLEEGDTFFFQPRPLKNLVLVDELDSLSPILFCQIADLANEDTPQLYVACGRGPRSSLRVLRHGLEVSEMAVSELPGNPNAVWTVRRHIEDEFDAYIIVSFVNATLVLSIGETVEEVTDSGFLGTTPTLSCSLLGDDALVQVYPDGIRHIRADKRVNEWKTPGKKTIVKCAVNQRQVVIALTGGELVYFEMDPSGQLNEYTERKEMSADVVCMSLANVPPGEQRSRFLAVGLVDNTVRIISLDPSDCLQPLSMQALPAQPESLCIVEMGGTEKQDELGERGSIGFLYLNIGLQNGVLLRTVLDPVTGDLSDTRTRYLGSRPVKLFRVRMQGQEAVLAMSSRSWLSYSYQSRFHLTPLSYETLEFASGFASEQCPEGIVAISTNTLRILALEKLGAVFNQVAFPLQYTPRKFVIHPESNNLIIIETDHNAYTEATKAQRKQQMAEEMVEAAGEDERELAAEMAAAFLNENLPESIFGAPKAGNGQWASVIRVMNPIQGNTLDLVQLEQNEAAFSVAVCRFSNTGEDWYVLVGVAKDLILNPRSVAGGFVYTYKLVNNGEKLEFLHKTPVEEVPAAIAPFQGRVLIGVGKLLRVYDLGKKKLLRKCENKHIANYISGIQTIGHRVIVSDVQESFIWVRYKRNENQLIIFADDTYPRWVTTASLLDYDTVAGADKFGNICVVRLPPNTNDEVDEDPTGNKALWDRGLLNGASQKAEVIMNYHVGETVLSLQKTTLIPGGSESLVYTTLSGGIGILVPFTSHEDHDFFQHVEMHLRSEHPPLCGRDHLSFRSYYFPVKNVIDGDLCEQFNSMEPNKQKNVSEELDRTPPEVSKKLEDIRTRYAF

SEQ ID NO: 3 - DNA, 3454 bp, Homo sapiens
  CDS (28)..(3438)
aagcttggtacggctagcgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagctcgacatggcgacggagcatcccgagcctcccaaagcagaattgcagctgccgccgccgccacctccaggccactatggcgcctgggctgcccaggagcttcaggccaagttggcagagatcggagctccgatccagggtaatcgcgaggagctggtggagcggctgcagagctacacccgccagactggcatcgtgctgaatcggccggttttgagaggggaagatggggacaaagccgctccacctcccatgtcggcacagctccctggaattcccatgccaccaccacctttgggactcccccctctgcagcctcctccgccacccccaccacctccaccaggccttggccttggctttcctatggcccacccaccaaatttggggcccccgcctcctctccgtgtgggtgagccagtggcactgtcagaggaggagcggctgaagttggctcagcagcaggcggcattgctgatgcagcaggaggagcgtgccaagcagcagggagatcattcgctgaaggaacatgagctcttggagcagcagaagcgggcagctgtgttactggagcaggaacgacagcaggagattgccaagatgggcaccccagtccctcggcccccacaagacatgggccagattggtgtgcgcactcctctgggtcctcgagtagctgctccagtgggcccagtgggccccactcctacagttttgcccatgggagcccctgttccccggcctcgtggtcccccaccgccccctggagatgagaacagagagatggatgacccctctgtgggccccaagatcccccaggctttggagaagatcctgcagctgaaggagagccgccaggaagagatgaattctcagcaggaggaagaggaaatggaaacagatgctcgctcgtccctgggccagtcagcgtcagagactgaggaggacacagtgtccgtatctaaaaaggagaaaaaccggaagcgtaggaaccgaaagaagaagaaaaagccccagcgggtgcgaggggtgtcctctgagagctctggggaccgggagaaagactcaacccggtcccgtggctctgattccccagcagctgatgttgagattgagtatgtgactgaagaacctgaaatttacgagcccaactttatcttctttaagaggatctttgaggcttttaagctcactgatgatgtgaagaaggagaaagagaaggagccagagaaacttgacaaactggagaactctgcagcccccaagaagaagggatttgaagaggagcacaaggacagtgatgatgacagcagtgatgacgagcaggaaaagaagccagaagcccccaagctgtccaagaagaagttgcgccgaatgaaccgcttcactgtggctgaactcaagcagctggtggctcggcccgatgtcgtggagatgcacgatgtgacagcgcaggaccctaagctcttggttcacctcaaggccactcggaactctgtgcctgtgccacgccactggtgttttaagcgcaaatacctgcagggcaaacggggcattgagaagccccccttcgagctgccagacttcatcaaacgcacaggcatccaggagatgcgagaggccctgcaggagaaggaagaacagaagaccatgaagtcaaaaatgcgagagaaagttcggcctaagatgggcaaaattgacatcgactaccagaaactgcatgatgccttcttcaagtggcagaccaagccaaagctgaccatccatggggacctgtactatgaggggaaggagttcgagacacgactgaaggagaagaagccaggagatctgtctgatgagctaaggatttccttggggatgccagtaggaccaaatgcccacaaggtccctcccccatggctgattgccatgcagcgatatggaccacccccatcgtatcccaacctgaaaatccctgggctgaactcgcccatccctgagagctgttcctttgggtaccatgctggtggctggggcaaacctccagtggatgagactgggaaaccgctctatggggacgtgtttggaaccaatgctgctgaatttcagaccaagactgaggaagaagagattgatcggaccccttggggggaactggaaccatctgatgaagaatcctcagaagaagaggaagaggaagaaagtgatgaagacaaaccagatgagacaggctttattacccctgcagacagtggccttatcactcctggaggcttttcatcagtgcctgctggaatggagacccctgaactcattgagctgaggaagaagaagattgaggaggcgatggacggaagtgagacacctcagctcttcactgtgttgccagagaagagaacagccactgttggaggggccatgatgggatcaacccacatttatgacatgtccacggttatgagccggaagggcccggctcctgagctgcaaggtgtggaagtggcgctggcgcctgaagagttggagctggatcctatggccatgacccagaagtatgaggagcatgtgcgggagcagcaggctcaagtagagaaggaggacttcagtgacatggtggctgagcacgctgccaaacagaagcaaaaaaaacggaaagctcagccccaggacagccgtgggggcagcaagaaatataaggagttcaagttttagaagcttaagtttaaac

SEQ ID NO: 4 - protein, 1136 aa, Homo sapiens [sequence omitted]

(19) United States
(12) Patent Application Publication
(10) Pub. No.: US 2009/0227795 A1
(43) Pub. Date: Sep. 10, 2009

(54) TARGET MOLECULES OF PLADIENOLIDES, COMPOUNDS BINDING TO SUCH TARGET MOLECULES, AND SCREENING METHOD THEREOF

(75) Inventors: Yoshihiko Kotake, Ibaraki (JP); Koji Sagane, Ibaraki (JP); Takashi Owa, Ibaraki (JP); Yoshiharu Mizui, Ibaraki (JP); Hajime Shimizu, Ibaraki (JP); Yuko Kiyosue, Hyogo (JP)

Correspondence Address: DARBY & DARBY P.C., P.O. BOX 770, Church Street Station, New York, NY, 10008-0770, US

(73) Assignee: Eisai R & D Management Co., Ltd., Tokyo (JP)

(21) Appl. No.: 12/375,516

(22) PCT Filed: Aug. 2, 2007
(86) PCT No.: PCT/JP2007/065574

§ 371 Date: Jan. 28, 2009

Related U.S. Application Data
(60) Provisional application No. 60/821,199, filed on Aug. 2, 2006.

(51) Int. Cl. C07D 405/14; G01N 33/566; C07D 409/14; C07F 5/02; C07D 407/06; A61P 35/00

(52) U.S. Cl. 544/374; 435/7.1; 548/303.7; 548/304.1; 548/405; 549/346

(57) ABSTRACT

A method of measuring the binding activity of a test compound to a splicing factor 3b (SF3b), which comprises the following steps of:
(a) contacting a labeled pladienolide compound and a test compound with a cell or a cell fraction; and
(b) measuring the distribution of the bound labeled compound.
   The method enables to screen for a novel active compound capable of acting on (binding to) a pladienolide target molecule or the like.

### CROSS REFERENCE TO PRIOR RELATED APPLICATIONS

This application is the U.S. national phase application under 35 U.S.C. § 371 of PCT International Application No. PCT/JP 2007/065574, filed Aug. 2, 2007, and claiming priority to U.S. provisional patent application No. 60/821,199 filed Aug. 2, 2006. International Application No. PCT/JP 2007/065574 published, in the Japanese language, as WO 2008/016187 A1 on Feb. 7, 2008 under PCT Article 21(2). The contents of all the priority applications are incorporated by reference, in their entireties, into the present disclosure.

### FIELD OF THE INVENTION

The present invention relates to target molecules of pladienolides and the derivatives thereof, compounds binding to the target molecules, and a screening method thereof.

### BACKGROUND

As a result of studies regarding anticancer agents conducted in recent years, novel kinase inhibitors have been successively found. Such kinase inhibitors contribute to the treatment of cancer patients or the improvement of QOL. However, cancer treatment results obtained with the use of anticancer agents have not yet been sufficient, and thus it is strongly desired that a novel anticancer agent be developed. In particular, the development of a novel anticancer agent based on a new drug-discovery target is expected, not only to treat cancer patients who cannot obtain sufficient therapeutic effects from the existing agents, but also to construct a new treatment strategy when it is used together with such existing agents.

It has been known that pladienolides exhibit excellent antitumor activity in vitro and in vivo (International Publications WO02/060890, WO03/099813, WO04/011661, and WO04/011459). It has been shown that pladienolides have anticancer spectra that completely differ from those of the existing antitumor agents, and suggested that they have novel action mechanisms. However, detailed action mechanisms such as in vivo target molecules have not yet been clarified.

### SUMMARY OF THE INVENTION

Isolation and identification of target molecules to which pladienolides bind result in the finding of a new drug-discovery target. Thus, the present inventors have designed and synthesized various probe compounds based on pladienolides, and they have made an attempt to identify their target molecules.

Objects of the present invention are to identify target molecules that exhibit the physiological activity of pladienolides, to provide probe compounds that are used in the above identification, and to provide a method of screening novel active compounds that act on (bind to) the target molecules of pladienolides, using newly identified target molecule compounds and/or probe-related compounds.

In the aforementioned background, the present inventors have conducted intensive studies directed towards identifying target molecules to which pladienolides specifically bind in vivo. As a result, the inventors have found that pladienolides bind to a factor known as SAP130 (which is also referred to as splicing factor 3b3 or SF3b3), so as to modify the functions thereof and the functions of a complex in which the SAP130 is a constituent, thereby exhibiting the physiological activity. Herein, SAP130 is a component of SF3b (splicing factor 3b, Will Cl et. al, EMBO, 2001, 20(16), 4536-46), and SF3b is a constitutional factor of U2 snRNP as a splicing machinery.

A compound that has an influence upon the functions of SF3b is:

(1) likely to have a great influence upon transcription in general based on the close relationship between splicing and transcription (Tom Maniatis & Robin Reed, Nature, 2002, 416, 499-507: Nick J. Proudfoot, Cell, 2002, 108, 501-512);

(2) SF3b is also considered to have an influence upon a cell cycle as it has been suggested that SF3b is associated with a cell cycle via the binding of SF3b to Cyclin E (Mol Cell Biol. 1998 August; 18(8): 4526-36).

From such assumptions, it is considered that SF3b is promising as a novel target of anticancer agents.

Moreover, using newly designed and synthesized probe compounds, novel physiologically active substances that bind to SAP130, SF3b, and U2snRNP can be searched. The inventors have found that antitumor agents that bind to such molecules can be screened, thereby completing the present invention.

That is, the present invention is as follows.

(1) A method of measuring the binding activity of a test compound to a splicing factor 3b (SF3b), which comprises the following steps of:
(a) contacting a labeled compound represented by the following formula (I) and a test compound with a cell or a cell fraction; and
(b) measuring the distribution of the bound labeled compound,

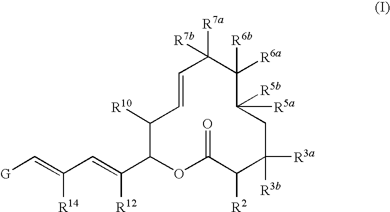

wherein, in the above formula (I),
R², R¹⁰, R¹², and R¹⁴ are the same as or different from one another and each represents hydrogen or methyl;
R^{3a}, R^{3b}, R^{5a}, R^{5b}, R^{6a}, and R^{6b} are the same as or different from one another and each represents,
(1) hydrogen,
(2) hydroxy,
(3) any one of the following groups, each of which may have substituent(s),
   (a) C₁₋₂₂ alkyl,
   (b) C₁₋₂₂ alkoxy,
   (c) ArCH₂O— (wherein Ar represents C₆₋₁₄ aryl or 5- to 14-membered ring heteroaryl, each of which may have substituent(s)),
   (d) C₂₋₂₂ acyloxy,
   (e) C₃₋₂₂ unsaturated acyloxy,
   (f) —OCOR^{CO} (wherein R^{CO} represents
      (i) C₆₋₁₄ aryl,
      (ii) 5- to 14-membered ring heteroaryl,
      (iii) C₁₋₂₂ alkoxy,
      (iv) unsaturated C₂₋₂₂ alkoxy,
      (v) C₆₋₁₄ aryloxy, or
      (vi) 5- to 14-membered ring heteroaryloxy, each of which may have substituent(s)),
   (g) C₁₋₂₂ alkylsulfonyloxy,
   (h) benzenesulfonyloxy, or
   (i) —OSiR^{S1}R^{S2}R^{S3} (wherein R^{s1}, R^{s2}, and R^{s3} are the same as or different from one another and each represents methyl, ethyl, i-propyl, t-butyl, or phenyl),
(4) halogen, or
(5) —R^{M}—NR^{N1}R^{N2} wherein R^{M} represents a single bond or —O—CO—; and R^{N1} and R^{N2} are as follows,
   1) R^{N1} and R^{N2} are the same as or different from each other and each represents
   (a) hydrogen or
   (b) any one of the following groups, each of which may have substituent(s):
      (i) C₁₋₂₂ alkyl,
      (ii) unsaturated C₃₋₂₂ alkyl,
      (iii) C₂₋₂₂ acyl,
      (iv) unsaturated C₃₋₂₂ acyl,
      (V) C₆₋₁₄ aryl,
      (vi) 5- to 14-membered ring heteroaryl,
      (vii) benzyl,
      (viii) C₁₋₂₂ alkylsulfonyl or
      (ix) benzenesulfonyl, or
   2) NR^{N1}R^{N2} may together represent 3- to 14-membered ring nitrogen-containing nonaromatic hetero ring, which may have substituent(s);
      R^{7a} and R^{7b} are as follows,
(1) R^{7a} and R^{7b} are different from each other and each independently represents,
   1) hydrogen,
   2) —OR^{H} wherein R^{H} represents hydrogen, methyl, or acetyl,
   3) —OR^{D} wherein R^{D} represents any one of the following groups, each of which may have substituent(s),
      (i) C₁₋₂₂ alkyl (in the case of methyl, it must have substituent(s)),
      (ii) —CH₂Ar,
      (iii) C₃₋₂₂ acyl,
      (iv) C₃₋₂₂ unsaturated acyl,
      (v) —COR^{CO},
      (vi) C₁₋₂₂ alkylsulfonyl,
      (vii) benzenesulfonyl or
      (viii) —SiR^{S1}R^{S2}R^{S3}, or
   4) —R^{M}—NR^{N1}R^{N2}, or
(2) R^{7a} and R^{7b} together represents
   1) a ketone structure (═O) or
   2) an oxime structure (═NOR^{OX}), wherein R^{OX} represents
      (a) C₁₋₂₂ alkyl,
      (b) unsaturated C₃₋₂₂ alkyl,
      (c) C₆₋₁₄ aryl,
      (d) 5- to 14-membered ring heteroaryl, or
      (e) benzyl, each of which may have substituent(s);

further,
R^{3a} and R^{3b} may together represent a ketone structure (═O) or an oxime structure (═NOR^{OX});

still further,
R^{6a} and R^{6b} may together represent a spiro-oxirane ring or exomethylene; and still further,
either R^{6a} or R^{6b} and either R^{7a} or R^{7b} may together form a 1,3-dioxolane ring;
   G represents,

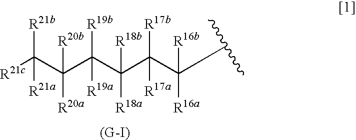

wherein R^{16a} and R^{16b} are the same as or different from each other and each represents hydrogen, methyl, or hydroxy;
R^{17a}, R^{17b}, R^{18a}, R^{18b}, R^{19a}, R^{19b}, R^{20a}, R^{20b}, R^{21a}, R^{21b} are the sane as or different from one another and each represents,

(1) hydrogen,

(2) methyl, which may have substituent(s),

(3) —OR^{H},

(4) —OR^{D},

(5) halogen, or

(6) —R^{M}—NR^{N1}R^{N2};

R^{21c} represents

(1) hydrogen or

(2)

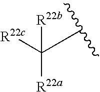

wherein R^{22a}, R^{22b}, and R^{22c} are the same as or different from one another and each represents
(a) hydrogen,
(b) methyl,
(c) hydroxy,
(d) —OR^{H},
(e) —OR^{D},
(f) —R^{M}—NR^{N1}R^{N2}, or
(g) halogen;
   further,
   either R^{18a} or R^{18b} and either R^{19a} or R^{19b} may together form a single bond, so as to represent a partial structure

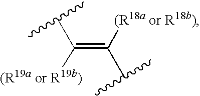

or they may bind to oxygen, so as to represent a partial structure

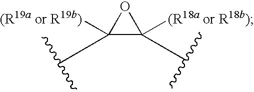

still further,
either R^{19a} or R^{19b} and either R^{20a} or R^{20b} may together form a single bond, so as to represent

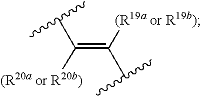

still further,
R^{21a} and R^{21b} may together represent (a) a ketone structure (═O) or (b) an oxime structure (═NOR^{OX});
still further,
either R^{21a} or R^{21b} and either R^{22a} or R^{22b} may together represent a partial structure

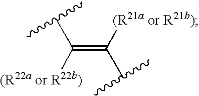

still further,
either R^{19a} or R^{19b} and either R^{21a} or R^{21b} may together represent a partial structure

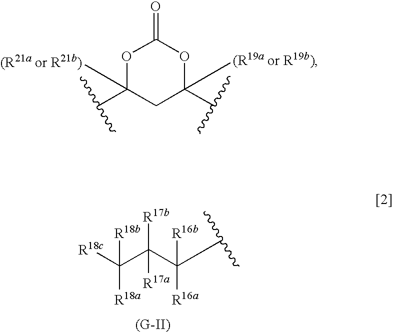

wherein R^{16a}, R^{16b}, R^{17a}, R^{17b}, R^{18a}, and R^{18b} have the same definitions as those described in formula (G-I); and R^{21c} represents (1) hydrogen or (2) the formula:

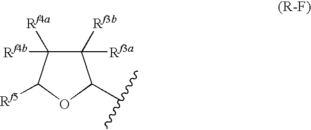

wherein R^{f3a}, R^{f3b}, R^{f4a}, and R^{f4b} are the same as or different from one another and each represents hydrogen, methyl, hydroxy, methoxy, or acetoxy, and R^{f5} represents methyl or ethyl or

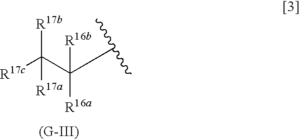

wherein R^{16a}, R^{16b}, R^{17a}, and R^{17b} have the same definitions as those described in formula (G-I); and R^{17c} represents (1) hydrogen or (2) the formula:

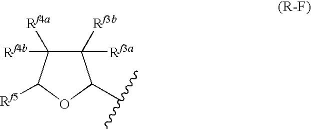

wherein R^{f3a}, R^{f3b}, R^{f4a}, and R^{f4b} are the same as or different from one another and each represents hydrogen, methyl, hydroxy, methoxy, or acetoxy, and R^{f5} represents methyl or ethyl.
(2) The method according to (1) above, wherein the compound represented by the above formula (I) is a compound represented by the following formula (IV):

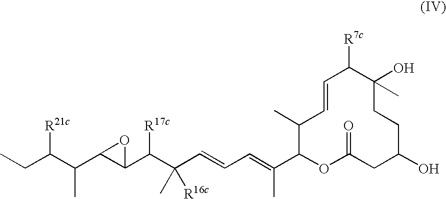

wherein, in the above formula (IV),
R^{16c}, R^{17c}, and R^{21c} are the same as or different from one another and each represents hydrogen, hydroxy, or methoxymethyl; and
R^{7c} represents hydroxy, acetoxy, or O—CO—NR^{N1′}R^{N2′} wherein R^{N1′} and R^{N2′} are the same as or different from each other and each represents hydrogen or C₁₋₆ alkyl.
(3) The method according to (1) above, wherein the compound represented by the above formula (I) is a compound represented by the following formula (V):

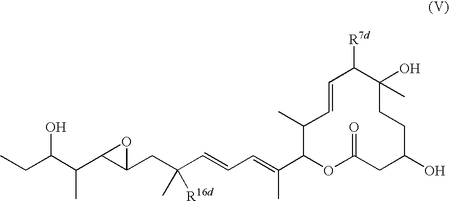

wherein, in the above formula (V),
R^{16d} represents hydrogen or hydroxy; and
R⁷d represents hydroxy, acetoxy, or O—CO—NHR^{N1′} (wherein R^{N1′} represents C₁₋₆ alkyl).
(4) The method according to any one of (1) to (3) above, wherein the activity of the test compound to bind to SAP130 in SF3b is measured.
(5) The method according to any one of (1) to (4) above, wherein the label is radioisotope label.
(6) The method according to any one of (1) to (4) above, wherein the label is fluorescent label.
(7) The method according to any one of (1) to (4) above, wherein the label is biotin label.
(8) The method according to any one of (1) to (4) above, wherein the label is a label with a compound that binds to a protein as a result of exposure to light.
(9) The method according to any one of (1) to (4) above, wherein, in the step (b), the amount of the labeled compound that is distributed in nuclei is measured, so as to measure the binding activity of the test compound to SF3 b.
(10) The method according to any one of (1) to (4) above, wherein, in the step (b), the amount of the labeled compound that is distributed in nuclear speckles is measured, so as to measure the binding activity of the test compound to SF3b.
(11) The method according to any one of (1) to (4) above, wherein, in the step (b), the pattern of distribution of the labeled compound in nuclear speckles is measured, so as to measure the binding activity of the test compound to SF3b.
(12) The method according to any one of (1) to (4) above, wherein, in the step (b), the amount of the labeled compound that is distributed in SF3b-bound state is measured, so as to measure the binding activity of the test compound to SF3b.
(13) The method according to any one of (1) to (4) above, wherein, in the step (b), the amount of the labeled compound that is distributed in SAP130-bound state is measured, so as to measure the binding activity of the test compound to SF3b.
(14) An anticancer agent binding to SF3b determined to have activity to bind to SF3b by the method according to any one of (1) to (13) above.
(15) An anticancer agent binding to SAP130 determined to have activity to bind to SAP130 by the method according to any one of (1) to (13) above.
(16) A labeled compound that is any of those represented by the following formula (II):

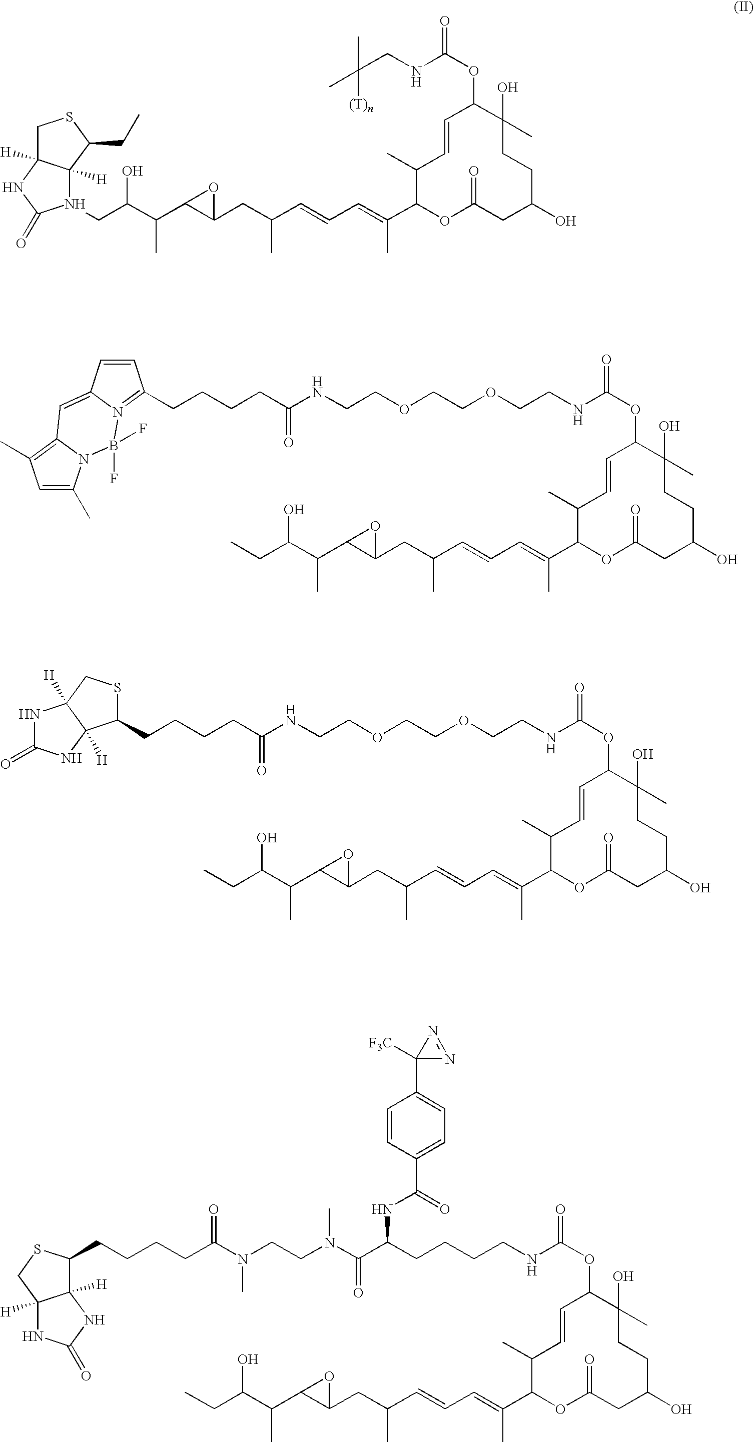

According to the present invention, a novel compound that acts on (binds to) the target molecules of pladienolides can be screened.

### BRIEF DESCRIPTION OF THE DRAWINGS

FIG. 1a is a view showing a ¹H-NMR spectrum.

FIG. 1b is a view showing ESI-MS.

FIG. 1c is a view showing the intracellular distribution of a tritium probe compound. The longitudinal axis of the graph indicates the value of radioactivity in the sample, which has been converted from the value of specific activity of the probe to the number of moles.

FIG. 1d is a view showing the intracellular distribution of a tritium probe compound (charcoal assay).

FIG. 2 is a view showing the results of a competition assay of pladieno analogues and pladieno derivatives with tritium probes (the correlation between the values of biological activity of competitors and competitive rates).

FIG. 3a is a view showing the observation results of intracellular localization of a fluorescent probe.

FIG. 3b is a view showing the observation results of intracellular localization of a fluorescent probe (the results of a competition assay of pladienolide compounds).

FIG. 4 is a view showing the experimental results of immunoprecipitation of a nuclear fraction prepared from cells treated with a tritium probe.

FIG. 5a is a view showing the results of detection of bound proteins by photoaffinity biotin probe treatment.

FIG. 5b is a view showing the results of detection of bound proteins by photoaffinity biotin probe treatment (the comparative results of the band positions of SAP155, SAP145, and SAP130).

FIG. 6a is a view showing the experimental results of detection of a band shift using GFP-fused SAP145.

FIG. 6b is a view showing the experimental results of detection of a band shift using GFP-fused SAP130.

FIG. 7 is a view showing the experimental results of a competition assay of pladieno compounds with photoaffinity biotin probes.

FIG. 8a is a view showing a representative example of fluorescent probe used in imaging analysis.

FIG. 8b is a view showing the results of imaging analysis.

### DETAILED DESCRIPTION OF THE INVENTION

The present invention will be described in detail below. The following embodiments are provided for illustrative purposes only, and are not intended to limit the scope of the present invention to such embodiments. The present invention can be carried out in various embodiments without departing from the spirit of the invention.

All publications, patent application publications, patents and other patent documents cited herein are incorporated herein by reference in their entirety. The present specification also incorporates the disclosures of US provisional application 6,821,199 based on which the present application claims priority.

In the present invention, the compound represented by the aforementioned formula (I) is related to a compound group, which is described in International Publications WO02/060890. WO03/099813, WO04/011661, and WO04/011459, and whose antitumor activity has been recognized. The above compound can be used as a compound to be labeled in the present invention. The above compound is referred to as “pladienolides” in the present specification, and it is also referred to as a “pladienolide analogue or derivative” at times. The compound represented by the following formula (IV) or (V) is a preferred example of such pladienolides.

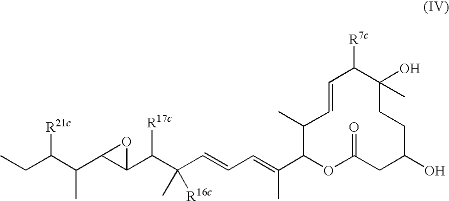

wherein R^{16c}, R^{17c} and R^{21c} are the same as or different from one another and each represents hydrogen, hydroxy or methoxymethyl;
R^{7c} represents hydroxy, acetoxy, O—CO—NR^{N1′}R^{N2′} (wherein R^{N1′} and R^{N2′} are the same as or different from each other and each represents hydrogen or C₁₋₆ alkyl)

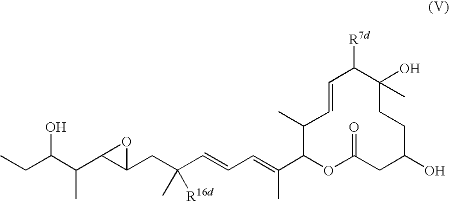

wherein R^{16d} represents hydrogen or hydroxy, preferably hydrogen;
R^{7d} represents hydroxy, acetoxy, O—CO—NHR^{N1′} (wherein R^{N1′} represents C₁₋₆ alkyl)

The position to be labeled is not limited, as long as antitumor activity is not lost due to such labeling. It is preferable that the acetyl group at position 7 be modified (labeled). Examples of a labeling method include labeling with a radioisotope and labeling with a fluorochrome (fluorophore), but examples are not limited thereto. In addition, a method of allowing biotin to bind to a molecule to be labeled, followed by detection using avidin that specifically binds to biotin, can also be adopted. However, such a specific bind is not limited to the case of detection with the combined use of biotin with avidin.

A structure activated by light irradiation to form a covalent bond together with surrounding functional groups (a photoaffinity moiety) may be introduced into a molecule to be labeled, enabling to prevent the labeled molecule from dissociation in a treatment with a protein denaturant, for example, by SDS, after a covalent bond was formed by light irradiation.

A method of synthesizing a labeled compound is specifically described in Examples A1 to A5.

In the present specification, the term “labeled compound” is used to mean a compound obtained by labeling the compound represented by the formula (I), and a preferred example of such a labeled compound is any compound represented by the formula (II).

An example of the screening, method of examining whether or not a test compound acts on (binds to) U2 snRNP, preferably SF3b, and more preferably SAP130 is a method including (a) contacting the labeled compound represented by the following formula (I) and a test compound with cells or a cell fraction, and (b) measuring distribution of the bound labeled compound. More specific examples are the following methods: It is to be noted that the term “contact” is used to mean that the labeled compound represented by the formula (I) and the test compound are allowed to exist with the cells or the cell fraction in a single reaction system or culture system. For example, a case where the labeled compound represented by the formula (I) and the test compound are added to a cell culture vessel, a case where the cells are cultured in the presence of the labeled compound represented by the formula (I) and the test compound, and a case where the labeled compound represented by the formula (I) and the test compound are mixed with a solution of the cell fraction, and the like are included.

1. Examination Method Utilizing Activity of Test Compound to Suppress Distribution of Labeled Pladienolides into Nuclei

(1) After cells have been cultured in the presence of a test compound and labeled pladienolides, the cells are fractionated into a nuclear fraction and other fractions. When the quantities of the labeled pladienolides in the nuclear fraction in the presence of the test compound become smaller than those in the absence of the test compound, for example, when the aforementioned quantities are 90% or less, preferably 70% or less, and more preferably 50% or less, it can be determined that the test compound has the activity of acting on (binding to) U2 snRNP, preferably SF3b, and more preferably SAP130.

In the present invention, after the cells have been fractionated into a nuclear fraction and other fractions, it is also possible that the test compound and the labeled pladienolides be added to each fraction, followed by incubation for a suitable period of time, and that the quantities of the labeled pladienolides in the nuclear fraction be then measured. In addition, the test compound and the labeled pladienolides may be simultaneously added to each fraction, or either the test compound or the labeled pladienolides may be previously added thereto. It is preferable that the test compound be added in advance.

As described in Example B2, there is a strong correlation between the antitumor activity of the test compound and the activity of the test compound to suppress distribution of the labeled pladienolides into a nuclear fraction. Accordingly, the method of the present invention makes it possible to screen a compound, which acts on (binds to) U2 snRNP, preferably SF3b, and more preferably SAP130, so as to exhibit antitumor activity.

(2) Distribution of the labeled pladienolides into nuclei can be examined by optically analyzing cells (for example, quantification of the labeled pladienolides distributed into the nuclei, on the basis of the image thereof, using a microscope). Thereafter, the result obtained by such optical analysis is used as an indicator, and a compound that suppresses distribution of the labeled pladienolides into the nuclei can be examined. An example of the method of examining distribution of the labeled pladienolides into the nuclei by image analysis will be described in Example B9.

2. Examination Method Utilizing Activity of Test Compound to Suppress Distribution of Labeled Pladienolides into Nuclear Speckles

Cells are cultured in the presence of a test compound and labeled pladienolides. The cells can be optically analyzed (for example, quantification of the labeled pladienolides distributed into nuclear speckles, on the basis of the image thereof, using a microscope), so as to examine distribution of the labeled pladienolides into the nuclear speckles. Thereafter, the result obtained by such optical analysis is used as an indicator, and a compound that suppresses distribution of the labeled pladienolides into the nuclear speckles can be examined.

Such nuclear speckles can be stained with an antibody reacting with a protein existing in the nuclear speckles, such as an anti-SC-35 antibody. As with the stained nuclear speckles, the labeled pladienolides distributed therein can be quantified, so as to measure distribution of the labeled pladienolides into the nuclear speckles.

When the quantities of the labeled pladienolides in the nuclear speckles in the presence of the test compound become smaller than those in the absence of the test compound, for example, when the aforementioned quantities are 90% or less, preferably 70% or less, and more preferably 50% or less, it can be determined that the test compound has the activity of acting on (binding to) U2 snRNP, preferably SF3b, and more preferably SAP130.

In addition, such cells are subjected to optical observation or image analysis, and based on the pattern of distribution of the labeled compound in the nuclear speckles, the binding activity of the test compound to SF3b can be measured. For example, when such nuclear speckles agglutinate and they are observed as enlarged nuclear speckles, it can be determined that the test compound has the activity of acting on (binding to) U2 snRNP, preferably SF3b, and more preferably SAP130.

As described in Example B3, there is a correlation between the antitumor activity of the test compound and the activity of the test compound to suppress distribution of the labeled pladienolides into nuclear speckles. Accordingly, the method of the present invention makes it possible to screen a compound, which acts on (binds to) U2 snRNP, preferably SF3b, and more preferably SAP130, so as to exhibit antitumor activity.

3. Examination Method Utilizing Activity of Test Compound to Suppress Binding of Labeled Pladienolides to U2snRNP, SF3b or SAP130

Cells are cultured in the presence of a test compound and labeled pladienolides, preferably photoaffinity labeled pladienolides. When such photoaffinity labeled pladienolides are used as labeled pladienolides, light is applied to the culture. After applying light, the cells are solubilized. Thereafter, proteins contained in the solubilized components are fractionated, preferably fractionated by SDS-PAGE, and the labeled pladienolides in a fraction that contains SAP130 are then quantified.

Preferably, light can be applied to an immunoprecipitation sample obtained by treating solubilized components obtained by solubilizing cells cultured in the presence of a test compound and photoaffinity labeled pladienolides with an anti-SAP155 antibody, an anti-SAP145 antibody, an anti-SAP120 antibody, an anti-U2B″ antibody or the like, and preferably with an anti-SAP155 antibody. Moreover, it is also possible that an immunoprecipitation sample obtained by treating solubilized components with an anti-SAP155 antibody, an anti-SAP145 antibody, an anti-SAP120 antibody, an anti-U2B″ antibody or the like, and preferably with an anti-SAP155 antibody, be treated with a test compound and photoaffinity labeled pladienolides, and that light be then applied to the resultant immunoprecipitation sample.

The wavelength used in light irradiation is not particularly limited. A wavelength that activates a used photoaffinity probe is preferable.

When the quantities of the labeled pladienolides in a U2 snRNP, SF3b, or SAP130 fraction in the presence of a test compound become smaller than those in the absence of the test compound, for example, when the aforementioned quantities are 90% or less, preferably 70% or less, and more preferably 50% or less, it can be determined that the test compound has the activity of acting on (binding to) U2 snRNP, preferably SF3b, and more preferably SAP130.

In the present invention, it is also possible that the test compound and the labeled pladienolides be added to the solubilized components after solubilizing the cells, followed by incubation for a suitable period of time, and that the quantities of the labeled pladienolides contained in the SAP130 fraction be then measured. In addition, the test compound and the labeled pladienolides may be simultaneously added to the solubilized components, or either the test compound or the labeled pladienolides may be previously added thereto. It is preferable that the test compound be added in advance.

SAP130 can be fused to a protein acting as a tag, such as GFP, so as to form a fused protein, and such a fused protein is then allowed to be expressed in certain cells. The use of such cells enables easy fractionation of SAP130, and thus it is preferable. Moreover, an immunoprecipitation experiment using an anti-GFP antibody is carried out on cells wherein SAP145 has been allowed to be expressed in the form of a fusion protein with a protein acting as a tag, such as GFP, so that SF3b can be fractionated.

As described in Example B7, there is a correlation between the antitumor activity of the test compound and the activity of the test compound to suppress distribution of the labeled pladienolides into the SAP130 fraction. Accordingly, the method of the present invention makes it possible to screen a compound, which acts on (binds to) SAP130 so as to exhibit antitumor activity.

A compound discovered by the present screening system has an effect on RNA splicing, and thus it is useful as an anticancer agent. Further, it is considered that this compound is also useful as a therapeutic agent for other diseases that are considered to be developed due to abnormal splicing, such as neurodegenerative diseases (e.g. familial Alzheimer's disease), dementia (e.g. frontotemporal dementia (Hutton, M. et al., Nature, 393:702-705, 1998)), mental disorders (e.g. familial dysautonomia (Hims M M et al., J Mol Med. 2007, 85(2):149-61. Epub 2007)), amyotrophic and myotonic degenerative diseases (e.g. spinal muscular atrophy and myotonic dystrophy), progeria, brain tumor, familial hypercholesterolemia, familial isolated growth hormone deficiency type II (Faustino, N. A. et al., Genes Dev., 17:419-437, 2006), and autoimmune disease. Moreover, it is also considered that the compound is further useful as a therapeutic agent for infectious diseases involving viruses that utilize splicing during their growth process, such as retrovirus (in particular HIV).

The compound of formula (I) is described below.

In the formula (I), R², R¹⁰, R¹², and R¹⁴ are the same as or different from one another and each represents hydrogen or methyl.

In the formula (I), R^{3a}, R^{3b}, R^{5a}, R^{5b}, R^{6a}, and R^{6b} are the same as or different from one another and each represents any one of (1) to (5) below:

(1) hydrogen,

(2) hydroxy,

(3) any one of the following groups, each of which may have substituent(s),
(a) C₁₋₂₂ alkyl,
(b) C₁₋₂₂ alkoxy,
(c) ArCH₂O— wherein Ar represents C₆₋₁₄ aryl or 5- to 14-membered ring heteroaryl, each of which may have substituent(s),
(d) C₂₋₂₂ acyloxy,
(e) C₃₋₂₂ unsaturated acyloxy,
(f) —OCOR^{CO} wherein R^{CO} represents
   (i) C₆₋₁₄ aryl,
   (ii) 5- to 14-membered ring heteroaryl,
   (iii) C₁₋₂₂ alkoxy,
   (iv) unsaturated C₂₋₂₂ alkoxy,
   (v) C₆₋₁₄ aryloxy, or
   (vi) 5- to 14-membered ring heteroaryloxy, each of which may have substituent(s),
(g) C₁₋₂₂ alkylsulfonyloxy,
(h) benzenesulfonyloxy, or
(i) —OSiR^{S1}R^{S2}R^{S3} wherein R^{s1}, R^{s2}, and R^{s3} are the same as or different from one another and each represents methyl, ethyl, i-propyl, t-butyl, or phenyl,

(4) halogen, or

(5) —R^{M}—NR^{N1}R^{N2} wherein R^{M} represents a single bond or —O—CO—.

In this case, R^{N1} and R^{N2} represent 1) or 2) as follows:
1) R^{N1} and R^{N2} are the same as or different from each other and each represents
(a) hydrogen or
(b) any one of the following groups, each of which may have substituent(s):
   (i) C₁₋₂₂ alkyl,
   (ii) unsaturated C₃₋₂₂ alkyl,
   (iii) C₂₋₂₂ acyl,
   (iv) unsaturated C₃₋₂₂ acyl,
   (V) C₆₋₁₄ aryl,
   (vi) 5- to 14-membered ring heteroaryl,
   (vii) benzyl,
   (viii) C₁₋₂₂ alkylsulfonyl or
   (ix) benzenesulfonyl, or
2) NR^{N1}R^{N2} may together represent 3- to 14-membered ring nitrogen-containing nonaromatic hetero ring, which may have substituent(s).

In the formula (I), R^{7a} and R^{7b} represent (1) or (2) below:
(1) R^{7a} and R^{7b} are different from each other and each independently represents,
   1) hydrogen,
   2) —OR^{H} wherein R^{H} represents hydrogen, methyl, or acetyl,
   3) —OR^{D} wherein R^{D} represents any one of the following groups, each of which may have substituent(s),
      (i) C₁₋₂₂ alkyl wherein in the case of methyl, it must have substituent(s),
      (ii) —CH₂Ar,
      (iii) C₃₋₂₂ acyl,
      (iv) C₃₋₂₂ unsaturated acyl,
      (v) —COR^{CO},
      (vi) C₁₋₂₂ alkylsulfonyl,
      (vii) benzenesulfonyl or
      (viii) —SiR^{S1}R^{S2}R^{S3}, or
4) —R^{M}—NR^{N1}R^{N2}, or

(2) R^{7a} and R^{7b} together represents
1) a ketone structure (═O) or
2) an oxime structure (═NOR^{OX}), wherein R^{OX} represents
   (a) C₁₋₂₂ alkyl,
   (b) unsaturated C₃₋₂₂ alkyl,
   (c) C₆₋₁₄ aryl,
   (d) 5- to 14-membered ring heteroaryl, or
   (e) benzyl, each of which may have substituent(s)).

In the formula (I), R^{3a} and R^{3b} may together represent a ketone structure (═O) or an oxime structure (═NOR^{OX}). Further, R^{6a} and R^{6b} may together represent a spiro-oxirane ring or exomethylene. Further, either R^{6a} or R^{6b} and either R^{7a} or R^{7b} may together form a 1,3-dioxolane ring.

In the formula (I), G represents any one of [1] to [3] below.

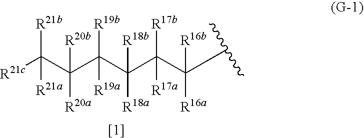

In the formula (G-I), R^{16a} and R^{16b} are the same as or different from each other and each represents hydrogen, methyl, or hydroxyl.

In the formula (G-I), R^{17a}, R^{17b}, R^{18a}, R^{18b}, R^{19a}R^{19b}, R^{20a}, R^{20b}, R^{21a}, and R^{21b} are the same as or different from one another and each represents any one of (1) to (6) below:

(1) hydrogen,

(2) methyl, which may have a substituent,

(3) OR^{H},

(4) —OR^{D},

(5) halogen, or

(6) —R^{M}—NR^{N1}R^{N2}.

In the formula (G-I), R^{21c} represents any one of (1) and (2) below:

(1) hydrogen or
(2)

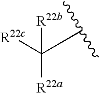

wherein R^{22a}, R^{22b}, and R^{22c} are the same as or different from one another and each represents

(a) hydrogen,

(b) methyl,

(c) hydroxy,

(d) —OR^{H},

(e) —OR^{D},

(f) —R^{M}—NR^{N1}R^{N2}, or

(g) halogen.

In the formula (G-I), further, either R^{18a} or R^{18b} and either R^{19a} or R^{19b} may together form a single bond, so as to represent a partial structure

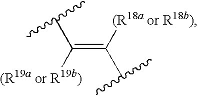

or they may bind to oxygen, so as to represent a partial structure

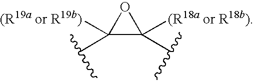

In the formula (G-I), still further, either R^{19a} or R^{19b} and either R^{20a} or R^{20b} may together form a single bond, so as to represent

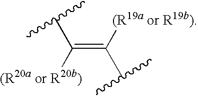

In the formula (G-I), still further, R^{21a} and R^{21b} may together represent (a) a ketone structure (═O) or (b) an oxime structure (═NOR^{OX}).
In the formula (G-I), still further, either R^{21a} or R^{21b} and either R^{22a} or R^{22b} may together represent a partial structure

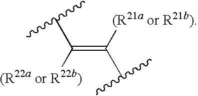

In the formula (G-I), still further, either R^{19a} or R^{19b} and either R^{21a} or R^{21b} may together represent a partial structure

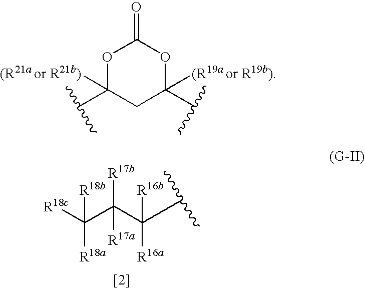

In the formula (G-II), R^{16a}, R^{16b}, R^{17a}, R^{17b}, R^{18a}, and R^{18b} have the same definitions as those described in formula (G-I).

In the formula (G-II), R^{18c} represents (1) or (2) below:

(1) hydrogen or

(2) the formula:

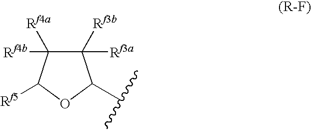

(wherein R^{f3a}, R^{f3b}, R^{f4a}, and R^{f4b} are the same as or different from one another and each represents hydrogen, methyl, hydroxy, methoxy, or acetoxy, and R^{f5} represents methyl or ethyl).

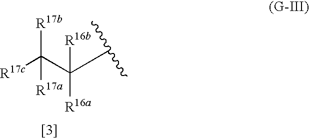

In the formula (G-III), R^{16a}, R^{16b}, R^{17a}, and R^{17b} have the same definitions as those described in formula (G-I).

In the formula (G-III), R^{17c} represents (1) or (2) below:

(1) hydrogen or

(2) the formula:

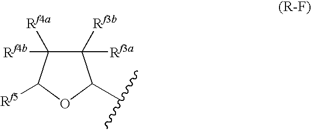

wherein R^{f3a}, R^{f3b}, R^{f4a}, and R^{f4b} are the same as or different from one another and each represents hydrogen, methyl, hydroxy, methoxy, or acetoxy, and R^{f5} represents methyl or ethyl.

The term “C₁₋₂₂ alkyl” used in the present specification means a linear or branched alkyl group or a cycloalkyl group having 1 to 22 carbon atoms, such as methyl group, ethyl group, n-propyl group, iso-propyl group, n-butyl group, iso-butyl group, sec-butyl group, tert-butyl group, n-pentyl group, 1,1-dimethylpropyl group, 1,2-dimethylpropyl group, 2,2-dimethylpropyl group, 1-ethylpropyl group, n-hexyl group, 1-ethyl-2-methylpropyl group, 1,1,2-trimethylpropyl group, 1-propylpropyl group, 1-methylbutyl group, 2-methylbutyl group, 1,1-dimethylbutyl group, 1,2-dimethylbutyl group, 2,2-dimethylbutyl group, 1,3-dimethylbuty group, 2,3-dimethylbutyl group, 2-ethylbutyl group, 2-methylpentyl group, 3-methylpentyl group, n-heptyl group, n-octyl group, n-nonyl group, n-decyl group, cyclopropyl group, cyclobutyl group, cyclopentyl group, or cyclohexyl group; preferably a linear or branched alkyl group or a cycloalkyl group having 1 to 6 carbon atoms, such as methyl group, ethyl group, n-propyl group, iso-propyl group, n-butyl group, iso-butyl group, sec-butyl group, tert-butyl group, or n-pentyl group; and more preferably, for example, methyl group, ethyl group, propyl group, iso-propyl group, n-butyl group, iso-butyl group, tert-butyl group, cyclopropyl group, cyclobutyl group, cyclopentyl group, or cyclohexyl group.

The term “unsaturated C₃₋₂₂ alkyl” used in the present specification means a linear or branched alkenyl group having 3 to 22 carbon atoms, or a linear or branched alkynyl group having 3 to 22 carbon atoms, such as allyl group, 1-propenyl group, isopropenyl group, 2-methyl-1-propenyl group, 2-methyl-2-propenyl group, 1-butenyl group, 2-butenyl group, 3-butenyl group, 1-pentenyl group, 1-hexenyl group, 1,3-hexanedienyl group, 1,5-hexanedienyl group, 1-propynyl group, 2-propynyl group, 1-butynyl group, 2-butynyl group, 3-butynyl group, 1-ethynyl-2-propynyl group, 2-methyl-3-propynyl group, 1-pentynyl group, 1-hexynyl group, 1-3-hexanediynyl group, or 1,5-hexanediynyl group; preferably a linear or branched alkenyl group having 3 to 10 carbon atoms, or a linear or branched alkynyl group having 3 to 10 carbon atoms, such as allyl group, 1-propenyl group, 2-propenyl group, isopropenyl group, 3-methyl-2-butenyl group, 3,7-dimethyl-2,6-octadienyl group, 1-propynyl group, 2-propynyl group, 1-butynyl group, 2-butynyl group, 3-butynyl group, or 3-methyl-1-propynyl group.

The term “C₁₋₂₂ alkoxy” used in the present specification means a group formed by binding an oxygen atom to the terminus of the above-defined “C₁₋₂₂ alkyl.” Examples of a suitable group include methoxy group, ethoxy group, n-propoxy group, iso-propoxy group, n-butoxy group, iso-butoxy group, sec-butoxy group, tert-butoxy group, n-pentyloxy group, iso-pentyloxy group, sec-pentyloxy group, n-hexoxy group, iso-hexoxy group, 1,1-dimethylpropyloxy group, 1,2-dimethylpropoxy group, 2,2-dimethylpropyloxy group, 1-methyl-2-ethylpropoxy group, 1-ethyl-2-methylpropoxy group, 1,1,2-trimethylpropoxy group, 1,2,2-trimethylpropoxy group, 1,1-dimethylbutoxy group, 1,2-dimethylbutoxy group, 2,2-dimethylbutoxy group, 2,3-dimethylbutyloxy group, 1,3-dimethylbutoxy group, 2-ethylbutoxy group, 2-methylpentoxy group, 3-methylpentoxy group, and hexyloxy group; preferably include, for example, methoxy group, ethoxy group, n-propoxy group, iso-propoxy group, iso-butoxy group, and 2,2-dimethylpropyloxy group.

The term “unsaturated C₂₋₂₂ alkoxy” used in the present specification means a group formed by binding an oxygen atom to the terminus of the above-defined “unsaturated C₃₋₂₂ alkyl,” vinyl, and ethyl. Examples of a suitable group include vinyloxy group, allyloxy group, 1-propenyloxy group, 2-propenyloxy group, isopropenyloxy group, 2-methyl-1-propenyloxy group, 2-methyl-2-propenyloxy group, 1-butenyloxy group, 2-butenyloxy group, 3-butenyloxy group, 1-pentenyloxy group, 1-hexenyloxy group, 1,3-hexanedienyloxy group, 1,5-hexanedienyloxy group, propargyloxy group, and 2-butynyloxy group; preferably include, allyloxy group, propargyloxy group, and 2-butynyloxy group.

The term “C₆₋₁₄ aryl” used in the present specification means an aromatic hydrocarbon cyclic group having 6 to 14 carbon atoms, and it includes a monocyclic group and a condensed ring such as a bicyclic group or a tricyclic group. Examples thereof are phenyl group, indenyl group, 1-naphthyl group, 2-naphthyl group, azulenyl group, heptalenyl group, indacenyl group, acenaphthyl group, fluorenyl group, phenalenyl group, phenanthrenyl group, and anthracenyl group; of which a preferred example is phenyl group, 1-naphthyl group, or 2-naphthyl group.

The term “5- to 14-membered heteroaryl” used in the present specification means a monocyclic, bicyclic or tricyclic, 5- to 14-membered aromatic heterocyclic group, which comprises one or more heteroatoms selected from the group consisting of a nitrogen atom, a sulfur atom and an oxygen atom. Examples of suitable group include a nitrogen-containing aromatic heterocylic group such as pyrrolyl group, pyridyl group, pyridazinyl group, pyrimidinyl group, pyrazinyl group, triazolyl group, tetrazolyl group, benzotriazolyl group, pyrazolyl group, imidazolyl group, benzimidazolyl group, indolyl group, isoindolyl group, indolizinyl group, purinyl group, indazolyl group, quinolyl group, isoquinolyl group, quinolizyl group, phthalazyl group, naphthyridinyl group, quinoxalyl group, quinazolinyl group, cinnolinyl group, pteridinyl group, imidazotriazinyl group, pyrazinopyridazinyl group, acridinyl group, phenanthridinyl group, carbazolyl group, carbazolinyl group, perimidinyl group, phenanthrolinyl group, phenazinyl group, imidazopyridinyl group, imidazopyrimidinyl group, pyrazolopyridinyl group, or pyrazolopyrimidinyl group; a sulfur-containing aromatic heterocyclic group such as thienyl group or benzothienyl group; an oxygen-containing aromatic heterocyclic group such as furyl group, pyranyl group, cyclopentapyranyl group, benzofuryl group, or isobenzofuryl group; an aromatic heterocyclic group containing different types of two or more heteroatoms such as thiazolyl group, isothiazolyl group, benzothiazolyl group, benzothiadiazolyl group, phenothiazinyl group, isoxazolyl group, furazanyl group, phenoxazinyl group, oxazolyl group, isoxazoyl group, benzoxazolyl group, oxadiazolyl group, pyrazolooxazolyl group, imidazothiazolyl group, thienofuranyl group, phlopyrrolyl group, or pyridoxadinyl group, of which a preferred example is thienyl group, furyl group, pyridyl group, pyridazinyl group, pyrimidyl group, or pyrazyl group.

The term “C₆₋₁₄ aryloxy” used in the present specification means a group formed by binding an oxygen atom to the terminus of the above-defined “C₆₋₁₄ aryl.” Specific examples include phenyloxy group, indenyloxy group, 1-naphthyloxy group, 2-naphthyloxy group, azulenyloxy group, heptalenyloxy group, indacenyloxy group, acenaphthyloxy group, fluorenyloxy group, phenalenyloxy group, phenanthrenyloxy group, and anthracenyloxy group, of which a preferred example is phenyloxy group, 1-naphthyloxy group, or 2-naphthyloxy group.

The term “5- to 14-membered heteroaryloxy” used in the present specification means a group formed by binding an oxygen atom to the terminus of the above-defined “5- to 14-membered heteroaryl.” Specific examples include pyrrolyloxy group, pyridyloxy group, pyridazinyloxy group, pyrimidinyloxy group, pyrazinyloxy group, triazolyloxy group, tetrazolyloxy group, benzotriazolyloxy group, pyrazolyloxy group, imidazolyloxy group, benzimidazolyloxy group, indolyloxy group, isoindolyloxy group, indolizinyloxy group, purinyloxy group, indazolyloxy group, quinolyloxy group, isoquinolyloxy group, quinolizyloxy group, phthalazyloxy group, naphthyridinyloxy group, quinoxalyloxy group, quinazolinyloxy group, cinnolinyloxy group, pteridinyloxy group, imidazotriazinyloxy group, pyrazinopyridazinyloxy group, acridinyloxy group, phenanthridinyloxy group, carbazolyloxy group, carbazolinyloxy group, perimidinyloxy group, phenanthrolinyloxy group, phenazinyloxy group, imidazopyridinyloxy group, imidazopyrimidinyloxy group, pyrazolopyrimidinyloxy group, pyrazolopyridinyloxy group, thienyloxy group, benzothienyloxy group, furyloxy group, pyranyloxy group, cyclopentapyranyloxy group, benzofuryloxy group, isobenzofuryloxy group, thiazolyloxy group, isothiazolyloxy group, benzothiazolyloxy group, benzothiadiazolyloxy group, phenothiazinyloxy group, isoxazolyloxy group, furazanyloxy group, phenoxazinyloxy group, oxazolyloxy group, isoxazoyloxy group, benzoxazolyloxy group, oxadiazolyloxy group, pyrazolooxazolyloxy group, imidazothiazolyloxy group, thienofuranyloxy group, phlopyrrolyloxy group, and pyridoxadinyloxy group, of which a preferred example is thienyloxy group, pyridyloxy group, pyrimidyloxy group, or pyrazyloxy group.

The term “C₂₋₂₂ acyl” used in the present specification means an acyl group with 2 to 22 carbon atoms. Examples of suitable groups include linear or branched acyl groups such as acetyl group, propionyl group, butyryl group, iso-butyryl group, valeryl group, iso-valeryl group, pivalyl group, caproyl group, decanoyl group, lauroyl group, myristoyl group, palmitoyl group, stearoyl group, and arachidoyl group.

Moreover, the term “C₂₋₂₂ acyloxy” used in the present specification means a group having a partial structure corresponding to the aforementioned “C₂₋₂₂ acyl.”

The term “unsaturated C₃₋₂₂ acyl” used in the present specification means an acyl group with 3 to 22 carbon atoms having double bond(s) or triple bond(s). Preferred unsaturated C₃₋₂₂ acyl groups include linear or branched acyl groups such as acryl group, propiol group, crotonyl group, iso-crotonyl group, oleinol group, and linolenoyl group.

Moreover, the term “unsaturated C₃₋₂₂ acyloxy” used in the present specification means a group having a partial structure corresponding to the aforementioned “unsaturated C₃₋₂₂ acyl.”

The term “C₁₋₂₂ alkylsulfonyl” used in the present specification means a group formed by binding the above-defined “C₁₋₂₂ alkyl” to sulfonyl. Specific examples include methylsulfonyl group, ethylsulfonyl group, n-propylsulfonyl group, and iso-propylsulfonyl group, of which a preferred example is methylsulfonyl group.

The term “C₁₋₂₂ alkylsulfonyloxy” used in the present specification means a group formed by binding an oxygen atom to the terminus of the above-defined “C₁₋₂₂ alkylsulfonyl,” such as methylsulfonyloxy group, ethylsulfonyloxy group, n-propylsulfonyloxy group, or iso-propylsulfonyloxy group, of which a preferred example is methylsulfonyloxy group.

The term “3- to 14-membered nitrogen-containing non-aromatic heterocyclic group” used in the present specification means a monocyclic, bicyclic or tricyclic 3- to 14-membered non-aromatic heterocyclic group that may comprise one or more heteroatoms selected from the group consisting of a nitrogen atom, a sulfur atom and an oxygen atom, as well as one nitrogen atom. Preferred examples include aziridinyl group, acetidyl group, pyrrolidinyl group, pyrrolyl group, piperidinyl group, piperazinyl group, imidazolyl group, pyrazolidyl group, imidazolidyl group, morpholyl group, thiomorpholyl group, imidazolinyl group, and oxazolinyl group. Moreover, the present non-aromatic heterocyclic groups further include groups induced from a pyridone ring and non-aromatic condensed rings (e.g. groups induced from a phthalimide ring, a succinimide ring, etc.).

The substituent of the expression “may have substituent(s)” used in the present specification means one or more groups selected from the group consisting of C₁₋₈ alkyl group, C₂₋₈ alkenyl group (e.g. vinyl group), C₂₋₈ alkynyl group (e.g. ethynyl group), C₆₋₁₄ aryl group (e.g. phenyl group, etc.), 5- to 14-membered heteroaryl group (e.g. thienyl group, furyl group, pyridyl group, pyridazyl group, pyrimidyl group, pyrazyl group, etc.), hydroxyl group, C₁₋₈ alkoxy group, C₁₋₈ acyl group, C₂₋₈ acyloxy group, C₂₋₈ alkenyloxycarbonyl group, C₂₋₈ alkynyloxycarbonyl group, C₁₋₈ alkoxycarbonyl group, halogen atom, hydroxycarbonyl group, thiol group, C₁₋₈ alkylthio group, C₁₋₈ alkylsulfoxide group, C₁₋₈ alkylsulfonyl group, C₁₋₈ alkylsulfonyloxy group, hydroxysulfonyl group, nitrile group, nitro group, nitroso group, amino group, N—C₁₋₈ alkylamino group, N,N-di-C₁₋₈ alkylamino group, N—C₂₋₈ alkenylamino group, N,N-di-C₂₋₈ alkenylamino group, N—C₂₋₈ alkynylamino group, N,N-di-C₂₋₈ alkynylamino group, N-arylamino group (e.g. phenylamino group), N-heteroarylamino group (e.g. 2-pyridylamino group, 3-pyridylamino group, 1-pyrroylamino group, etc.), N—C₁₋₈ alkyl-N-arylamino group, N—C₁₋₈ alkyl-N-heteroarylamino group, aralkyloxy group, heteroaryloxy group, C₁₋₈ alkylsulfonylamino group, C₂₋₈ alkenylsulfonylamino group, C₂₋₈ alkynylsulfonylamino group, N—C₁₋₈ alkylcarbamoyl group, N—C₁₋₈ alkylcarbamoyl group, N—C₂₋₈ alkenylcarbamoyl group, N,N-di-C₂₋₈ alkynylcarbamoyl group, C₂₋₈ acylamino group, etc.

The present invention will be more specifically described in the following examples. However, these examples are not intended to limit the scope of the present invention.

In the following examples, pladienolides A, B, D and E7170 are as follows:

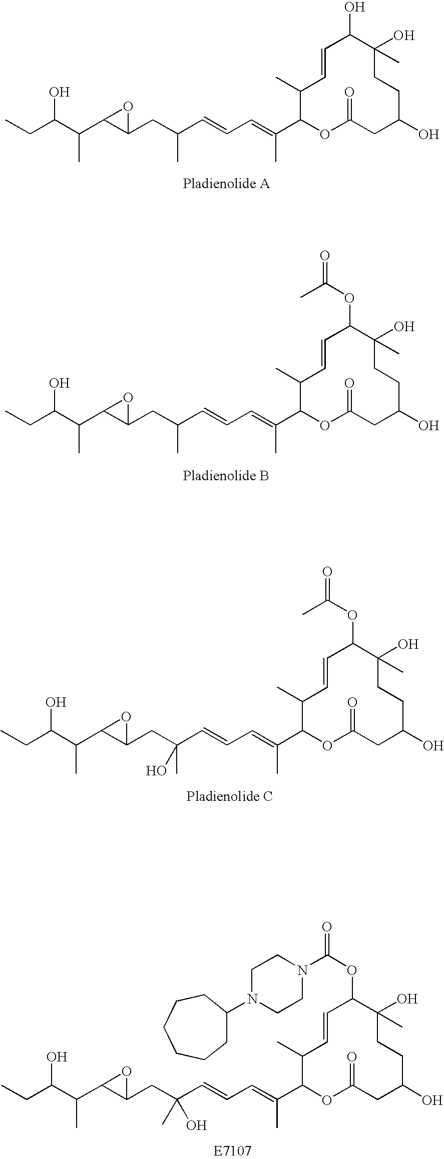

The labeled compound of the present invention was synthesized by the following method.

### Example A1

### (8E,12E,14E)-7-(N—[G-³H]ethylcarbamoyloxy)-3,6,21-trihydroxy-6,10,12,16,20-pentamethyl-18,19-epoxytricosa-8,12,14-trien-11-olide

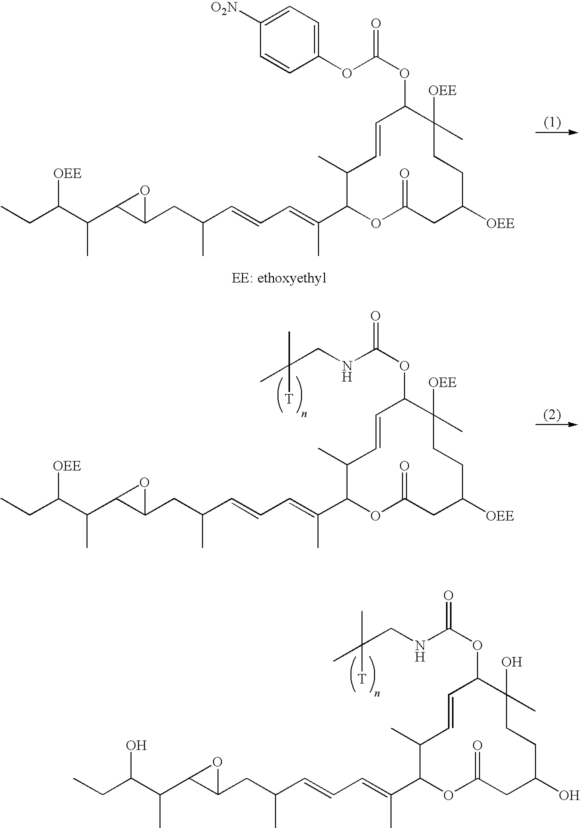

### (1) (8E,12E,14E)-7-(N—[G-3H]ethylcarbamoyloxy)-3,6,21-tri(1-ethoxyethoxy)-6,10,12,16,20-pentamethyl-18,19-epoxytricosa-8,12,14-trien-11-olide

(8E,12E,14E)-3,6,21-tri(1-ethoxyethoxy)-6,10,12,16,20-pentamethyl-7-(4-nitrophenoxy)carboxy-18,19-epoxytricosa-8,12,14-trien-11-olide (15 mg) described in Example B44 of the Patent Document (WO02/060890) was dissolved in anhydrous THF (1.5 ml). Thereafter, [G-³H]ethylamine (8.7 Ci) was distilled and was then added dropwise to the reaction solution. The reaction solution was stirred at room temperature for 24 hours, and the solvent was then distilled away under reduced pressure. The residue was dissolved in a small amount of ethyl acetate (yield: 680 mCi). The obtained solution was purified by silica gel HPLC (elution solvent: n-hexane:ethyl acetate=1:4 to 1:1), so as to obtain the titled compound (yield: 113 mCi).

### (2) (8E,12E,14E)-7-(N—[G-3H]ethylcarbamoyloxy)-3,6,21-trihydroxy-6,10,12,16,20-pentamethyl-18,19-epoxytricosa-8,12,14-trien-11-olide

(8E,12E,14E)-7-(N—[G-3H]ethylcarbamoyloxy)-3,6,21-tri(1-ethoxyethoxy)-6,10,12,16,20-pentamethyl-18,19-epoxytricosa-8,12,14-trien-11-olide (113 mCi) and pyridinium p-toluenesulfonate (0.75 my) were dissolved in methanol (1 ml). The reaction solution was stirred at room temperature for 4.5 hours. Thereafter, the solvent was evaporated from the reaction solution under nitrogen gas flow, and the obtained residue was then dissolved in ethyl acetate. The obtained solution was passed through a silica gel short column, so as to obtain a roughly purified product (105 mCi).

The roughly purified product was purified by silica gel HPLC (elution solvent: n-hexane:2-propanol=6:4). Thereafter, fractions of interest were combined, and the solvent was then distilled away. The residue was desiccated and was then dissolved in ethanol (105 mCi). Then, an ethanol solution that contained the product of interest of 1 mCi/ml was prepared, and it was then stored.

TOF-MS m/z 590, 592 (main peak), 594 (M+Na)⁺

Radiochemical purity: 97.0% (by HPLC), 97% (by TLC)

Specific activity: 56 Ci/mmol (by Mass Spectroscopy)

### Example A2

### 7-(N-(2-(2-(2-(N-(5-(5,5-difluoro-7,9-dimethyl-5H-dipyrrolo[1,2-c:2′,1′-f][1,3,2]diazaborinin-4-ium-5-uid-3-yl)pentanoyl)amino)ethoxy)ethoxy)ethyl)carbamoyloxy)-3,6,21-trihydroxy-6,10,12,16,20-pentamethyl-18,19-epoxytricosa-8,12,14-trien-11-olide

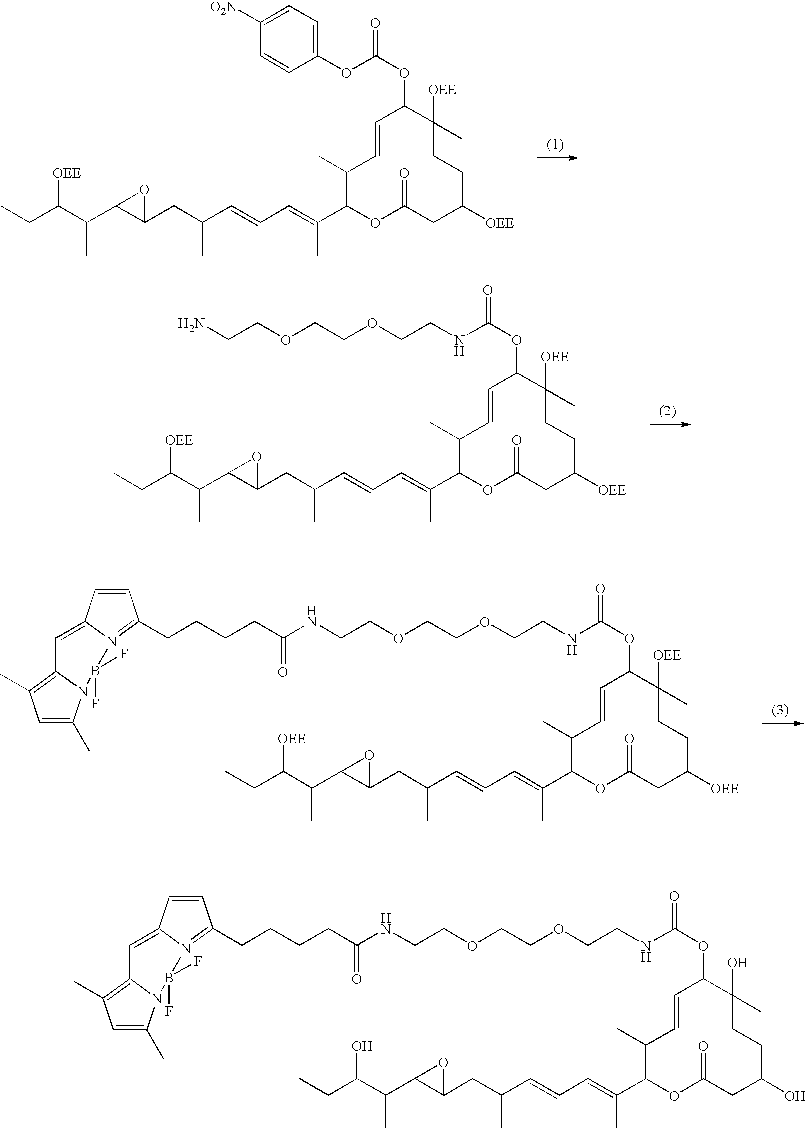

### (1) (8E,12E,14E)-7-(N-(2-(2-(2-aminoethoxy)ethoxy)ethyl)carbamoyloxy)-3,6,21-tris(1-ethoxyethoxy)-6,10,12,16,20-pentamethyl-18,19-epoxytricosa-8,12,14-trien-11-olide

1,8-diamino-3,6-dioxaoctane (127 mg, 0.85 mmol) was dissolved in THF (5 ml). Thereafter, a THF solution (15 ml) of (8E,12E,14E)-3,6,21-tri(1-ethoxyethoxy)-6,10,12,16,20-pentamethyl-7-(4-nitrophenoxy)carboxy-18,19-epoxytricosa-8,12,14-tri en-11-olide (150 mg, 0.17 mmol) described in Example B44 of the Patent Document (WO02/060890) was added dropwise to the obtained solution. The reaction solution was stirred at room temperature for 3 hours, and the solvent was then distilled away under reduced pressure. The obtained residue was purified by silica gel column chromatography (Fuji Silysia; NH silica gel; CH₂Cl₂:MeOH=10:1), so as to obtain the titled compound (0.10 g, 66%) in the form of a light yellow oil product.

ESI-MS m/z 885 (M+H)⁺.

### (2) 7-(N-(2-(2-(2-(N-(5-(5,5-difluoro-7,9-dimethyl-5H-dipyrrolo[1,2-c:2′,1′-t][1,3,2]diazaborinin-4-ium-5-uid-3-yl)pentanoyl)amino)ethoxy)ethoxy)ethyl)carbamoyloxy)-3,6,21-tri(1-ethoxyethoxy)-6,10,12,16,20-pentamethyl-18,19-epoxytricosa-8,12,14-trien-11-olide

(8E,12E,14E)-7-(N-(2-(2-(2-aminoethoxy)ethoxy)ethyl)carbamoyloxy)-3,6,21-tris(1-ethoxyethoxy)-6,10,12,16,20-pentamethyl-18,19-epoxytricosa-8,12,14-trien-11-olide (10.6 mg, 12 μmol) and 4,4-difluoro-5,7-dimethyl-4-bora-3a,4a-diaza-s-indacene-3-pentanoic acid, succinimidyl ester (Molecular Probes; 5 mg, 12 μmol) were dissolved in THF (3 ml). The reaction solution was stirred at room temperature for 12 hours. Thereafter, the solvent was distilled away under reduced pressure, and the residue was then purified by silica gel column chromatography (Kanto Chemical, spherical, neutral, CH₂Cl₂:MeOH=10:1), so as to obtain the captioned compound (13.1 mg, 92%) in the form of an orange oil product.

ESI-MS m/z 1210 (M+Na)⁺.

### (3) 7-(N-(2-(2-(2-(N-(5-(5,5-difluoro-7,9-dimethyl-5H-dipyrrolo[1,2-c:2′,1′-f][1,3,2]diazaborinin-4-ium-5-uid-3-yl)pentanoyl)amino)ethoxy)ethoxy)ethyl)carbamoyl oxy)-3,6,21-trihydroxy-6,10,12,16,20-pentamethyl-18,19-epoxytricosa-8,12,14-trien-11-olide

7-(N-(2-(2-(2-(N-(5-(5,5-difluoro-7,9-dimethyl-5H-dipyrrolo[1,2-c:2′,1′-f][1,3,2]diazaborinin-4-ium-5-uid-3-yl)pentanoyl)amino)ethoxy)ethoxy)ethyl)carbamoyloxy)-3,6,21-tri(1-ethoxyethoxy)-6,10,12,16,20-pentamethyl-18,19-epoxytricosa-8,12,14-trien-11-olide (13 mg, 11 μmol), methanol (4 ml), and pyridinium p-toluenesulfonate (2 mg, 8 μmol) were dissolved in methanol (4 ml). The reaction solution was stirred at room temperature for 24 hours. Thereafter, the solvent was distilled away under reduced pressure, and the residue was then dissolved in ethyl acetate. The obtained solution was sequentially washed with distilled water, a sodium bicarbonate aqueous solution, and a saline solution. Thereafter, the organic layer was dried over magnesium sulfate, and the solvent was then distilled away. The obtained residue was purified by preparative TLC (Merck Art. 105628; CH₂Cl₂:MeOH=10:1), so as to obtain the titled compound (9.3 mg, 87%) in the form of an orange solid.

¹H-NMR

Spectrum (CD₃OD, 600 MHz) δ(ppm): 0.91 (3H, d, J=6.8 Hz), 0.94 (3Hd, J=7.0 Hz), 0.98 (3H, t, J=7.5 Hz), 1.12 (3H, d, J=6.8 Hz), 1.18-1.20 (4H, m), 1.30-1.70 (8H, m), 1.72-1.84 (7H, m), 2.28-2.34 (5H, m), 2.46-2.64 (7H, m), 2.69 (1H, dd, J=2.1, 8.1 Hz), 2.76 (1H, dt, Jd=2.4 Hz, Jt=6.0H z), 2.82 (2H, br), 2.98 (2H, t, J=7.3 Hz), 3.32 (1H, t, J=5.5 Hz), 3.38-3.42 (2H, m), 3.52-3.60 (5H, m), 3.62 (4H, s), 3.78-3.84 (1H, m), 4.94 (1H, d, J=9.0 Hz), 5.07 (1H, d, J=10.7 Hz), 5.59 (1H, dd, J=10.0, 15.2 Hz), 5.69 (1H, dd, 8.4, 15.1 Hz), 5.74 (1H, dd, J=10.0, 15.1 Hz), 6.12 (1H, d, J=11.1 Hz), 6.23 (1H, s), 6.35 (1H, dd, J=10.9, 15.2 Hz), 6.40 (1H, d, 4.1 Hz), 7.07 (1H, d, J=3.8 Hz), 7.45 (1H, s); ESI-MS m/z 993 (M+Na)⁺.

### Example A3

### (8E,12E,14E)-3,6,21-trihydroxy-6,10,12,16,20-pentamethyl-7-(N-(2-(2-(2-(N-(5-((3aS,4S,6aR)-2-oxohexahydro-1H-thieno[3,4-d]imidazol-4-yl)pentanoyl)amino)ethoxy)ethoxy)ethyl)carbamoyloxy)-18,19-epoxytricosa-8,12,14-trien-11-olide

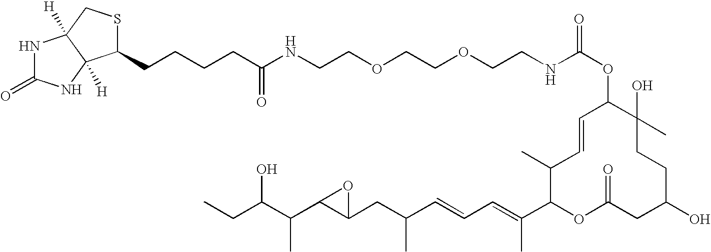

The titled compound (8 mg) was obtained in the form of a colorless oil product by the same method as that described in Example A2 with the exception that N-(+)biotinyl-1,8-diamino-3,6-dioxaoctane (Pierce; Biotin PEO-Amine) was used instead of 1,8-diamino-3,6-dioxaoctane.

¹H-NMR

Spectrum (CD₃OD, 500 MHz) δ(ppm): 0.89 (3H, d, J=5.5 Hz), 0.91 (3H, d, J=7.0 Hz), 0.94 (3H, t, J=7.5 Hz), 1.09 (3H, d, J=6.8 Hz), 1.16-1.26 (4H, tm), 1.27-1.80 (17H, m), 2.22 (2H, t, J=7.3 Hz), 2.42-2.64 (4H, m), 2.66 (1H, dd, J=2.2, 8.1 Hz) 2.68-2.76 (2H, m), 2.93 (1H, dd, J=4.6, 12.9 Hz), 2.98-3.25 (1H, m), 3.28-3.34 (2H, m, covered with CD₃OD), 3.36 (2H, t, J=5.6 Hz), 3.49-3.58 (5H, m), 3.62 (4H, s), 3.75-3.82 (1H, m), 4.31 (1H, dd, J=4.4, 7.8 Hz), 4.49 (1H, dd, J=4.9.7.8 Hz), 4.90 (1H, d, J=9.8 Hz), 5.05 (1H, d, J=10.7 Hz), 5.56 (1H, dd, J=9.5, 15.4 Hz), 5.63-5.78 (2H, m), 6.09 (1H, d, J=10.7 Hz), 6.32 (1H, dd, J=11.0, 9, 15.1 Hz); ESI-MS m/z 917 (M+Na)⁺, 929 (M+Cl)⁻

### Example A4

### N-((1S)-5-amino-1-(methyl(2-(methyl(5-((3aS,4S,6aR)-2-oxohexahydro-1H-thieno[3,4-d]imidazol-4-yl)pentanoyl)amino)ethyl)carbamoyl)pentyl)-4-(3-(trifluoromethyl)-3H-diaziren-3-yl)benzamide

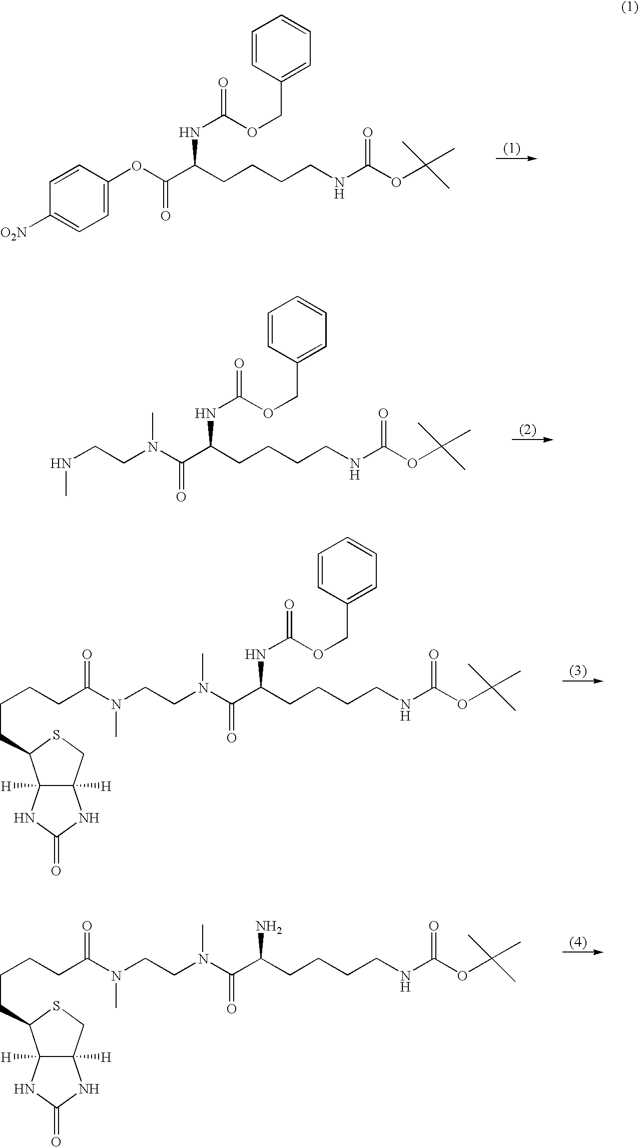

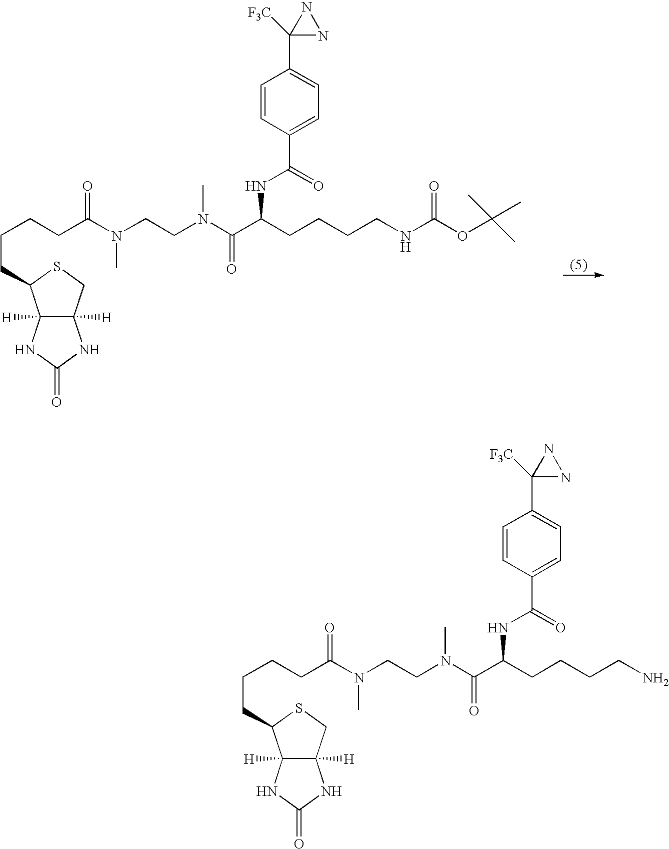

### Benzyl-((1S)-5-((tert-butoxycarbonyl)amino)-1-(methyl(2-(methylamino)ethyl)carbamoyl)pentyl)carbamate

1,4-dimethylethylenediamine (0.88 g, 10 mmol) was dissolved in THF (10 ml). While the obtained solution was stirred at room temperature, commercially available Z-Lys (BOC)-ONP (1 g, 2 mmol) was added thereto, dividedly several times. The reaction solution was stirred at room temperature for 12 hours. Thereafter, the solvent was distilled away, and using ethyl acetate, the residue was partitioned between oil and water. The organic layer was sequentially washed with distilled water and a saline solution, and it was then dried over magnesium sulfate. Thereafter, the solvent was distilled away. The obtained residue was purified by silica gel column chromatography (Fuji Sylysia; NH silica gel; AcOEt:MeOH=10:1), so as to obtain the titled compound (900 mg, quant.) in the form of a colorless oil product.

¹H-NMR

Spectrum (CD₃Cl₃, 400 MHz) δ(ppm): 1.30-1.78 (15H, m), 2.38-2.48 (3H, m), 2.68-2.88 (2H, m), 2.96 (1H, s), 3.00-3.16 (4H, br-m), 3.32-3.60 (2H, m), 4.58-4.76 (2H, m), 5.09 (3H, s), 5.60 (1H, d, J=8.8 Hz), 5.70 (1H, d, J=8.0 Hz), 7.28-7.40 (5H, m); ESI-MS m/z 473 (M+Na)⁺.

### (2) Benzyl-((1S)-5-((tert-butoxycarbonyl)amino)-1-(methyl(2-(methyl(5-((3aS,4S,6aR)-2-oxohexahydro-1H-thieno[3,4-d]imidazol-4-yl)pentanoyl)amino)ethyl)carbamoyl)pentyl)carbamate

The product (0.9 g, 2 mmol) obtained in (1) above was dissolved in THF-DMF (20 ml-10 ml). While the obtained solution was stirred at room temperature, biotin N-hydroxysuccinimide (0.7 g, 2 mmol) was added thereto, dividedly several times. The reaction solution was stirred at room temperature for 12 hours. Thereafter, the solvent was distilled away, and using ethyl acetate, the residue was partitioned between oil and water. The organic layer was sequentially washed with distilled water and a saline solution, and it was then dried over magnesium sulfate. Thereafter, the solvent was distilled away. The obtained residue was purified by silica gel column chromatography (Merck Art. 1.09385; CH₂Cl₂:MeOH=10:1), so as to obtain the titled compound (1.2 g, 89%) in the form of a colorless amorphous product.

¹H-NMR

Spectrum (DMSO-d₆, 400 MHz) δ(ppm): 1.10-1.65 (21H, m), 2.05-3.65 (17H, m), 4.04-4.15 (1H, m), 4.22-4.40 (2H, m), 4.92-5.06 (2H, m), 6.30-6.48 (2H, m), 6.70-6.80 (1H, m), 7.22-7.44 (6H, m); ESI-MS m/z 699 (M+Na)⁺.

### (3) tert-butyl-((5S)-5-amino-6-(methyl(2-(methyl(5-((3aS,4S,6aR)-2-oxohexahydro-1H-thieno[3,4-d]imidazol-4-yl)pentanoyl-9-amino)ethyl)amino)-6-oxohexyl)carbamate

The product (1.2 g, 1.8 mmol) obtained in (2) above was dissolved in MeOH (20 ml-10 ml). A 7 M ammonia/methanol solution (20 ml) was added to the reaction solution. Thereafter, 10% palladium carbon (0.83 g) was suspended in the obtained reaction solution, followed by a catalytic reduction reaction using a hydrogenation apparatus. The yield of the reaction product was confirmed with TLC (Merck Art. 1.05719; CH₂Cl₂:MeOH=10:1), and the catalyst was then removed by filtration. Thereafter, the filtrate was distilled away under reduced pressure. The obtained residue was desiccated under reduced pressure using a desiccator, so as to obtain the titled compound (0.95 g, 98%) in the form of a light yellow amorphous product.

¹H-NMR

Spectrum (DMSO-d₆, 400 MHz) δ(ppm): 1.05-1.70 (21H, m), 2.10-2.40 (2H, m), 2.50-2.65 (1H, m), 2.74-3.66 (15H, m), 4.06-4.20 (1H, m), 4.26-4.36 (1H, m), 6.336 (1H, br-s), 6.43 (1H, br-s), 6.70-6.84 (1H, m); NH₂ was not detected as a clear peak; ESI-MS m/z 565 (M+Na)⁺.

### (4) tert-butyl-((5S)-6-(methyl(2-(methyl(5-((3aS,4S,6aR)-2-oxohexahydro-1H-thieno[3,4-d]imidazol-4-yl)pentanoyl)amino)ethyl)amino)-6-oxo-5-((4-(3-(trifluoromethyl)-3H-diaziren-3-yl)benzoyl)amino)hexyl)carbamate

The product (0.95 g, 1.75 mmol) obtained in (3) above and 4-(3-(trifluoromethyl)-3H-diaziren-3-yl)benzoic acid (0.44 g, 1.93 mmol) synthesized in accordance with the publication (Nassal M. Liebigs Ann. Chem. 1983, 1510-1523) were dissolved in THF-DMF (20 ml-20 ml). A condensing agent, 4-(4,6-dimethoxy[1.3.5]triazin-2-yl)-4-methyl morpholium chloride hydrate (Wako; 1.05 g; 3.45 mmol), was added to the reaction solution, dividedly several times. Thereafter, the obtained reaction solution was stirred at room temperature for 24 hours. The solvent was distilled away, and using ethyl acetate, the residue was partitioned between oil and water. The organic layer was sequentially washed with distilled water and a saline solution, and it was then dried over magnesium sulfate. Thereafter, the solvent was distilled away. The obtained residue was purified by silica gel column chromatography (Merck Art. 1.09385; CH₂Cl₂:MeOH=20:1 to 8:1), so as to obtain the captioned compound (1.18 g, 89%) in the form of a colorless amorphous product.

¹H-NMR

Spectrum (DMSO-d₆, 400 MHz) δ(ppm): 1.05-1.70 (21H, nm), 2.10-2.38 (2H, m), 2.44-2.62 (11H, m), 2.70-3.80 (14H, m), 4.04-4.20 (1H, m), 4.24-4.36 (1H, m), 4.70-4.90 (1H, m), 6.30-6.58 (2H, m), 6.70-6.84 (1H, m), 7.37 (2H, d, J=8.0 Hz), 7.94-8.06 (2H, m), 8.64-8.96 (1H, m); ESI-MS m/z 777 (M+Na)⁺.

### (5) N-((1S)-5-amino-1-(methyl(2-(methyl(5-((3aS,4S,6aR)-2-oxohexahydro-1H-thieno[3,4-d]imidazol-4-yl)pentanoyl)amino)ethyl)carbamoyl)pentyl)-4-(3-(trifluoromethyl)-3H-diaziren-3-yl)benzamide

The product (0.29 g, 0.38 mmol) obtained in (4) above was dissolved in ethyl acetate (20 ml), and the obtained solution was then stirred under cooling on ice. Thereafter, a 4 M hydrogen chloride-ethyl acetate solution (5 ml) was added to the obtained reaction solution, and the obtained mixture was then stirred at room temperature for 12 hours. The solvent was distilled away, and using methylene chloride and ammonia water, the residue was partitioned between oil and water. Further, the water layer was extracted again with methylene chloride. The obtained organic layers were combined. The resultant was washed with a saline solution, and it was then dried over magnesium sulfate. Thereafter, the solvent was distilled away, so as to obtain the titled compound (0.21 g, 84%) in the form of a colorless amorphous product.

ESI-MS m/z 655 (M+H)⁺, 689 (M+Cl)⁻

### Example A5

### (8E,2E,14E)-3,621-trihydroxy-6,10,12,16,20-pentamethyl-7-(N-((5S)-6-(N-methyl-N-(2-(N-methyl-N-(5-((3aS,4S,6aR)-2-oxohexahydro-1H-thieno[3,4-d]imidazol-4-yl)pentanoyl)amino)ethyl)amino)-6-oxo-5-(N-(4-(3-(trifluoromethyl)-3H-diaziren-3-yl)benzoyl)amino)hexyl)carbamoyloxy)-18,19-epoxytricosa-8,12,14-trien-11-olide

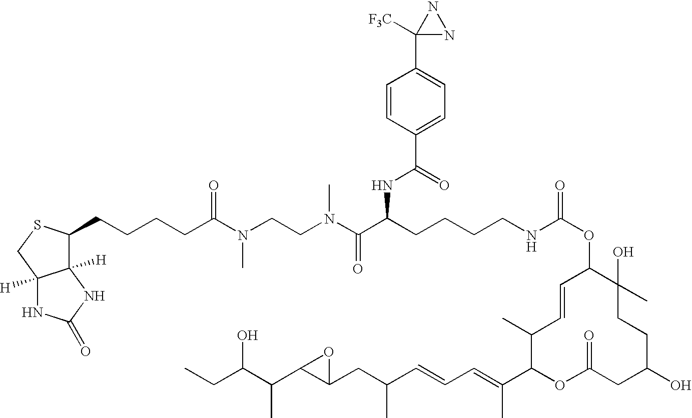

The titled compound (40 mg, 25%) was obtained in the form of a colorless oil product by the same method as that described in Example A2 using the amine compound synthesized in Example A4.

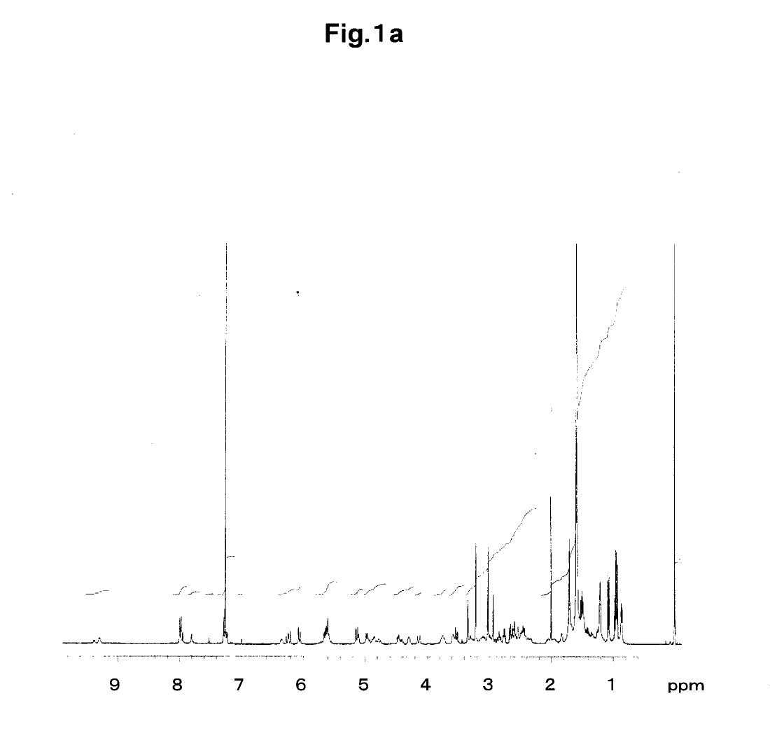

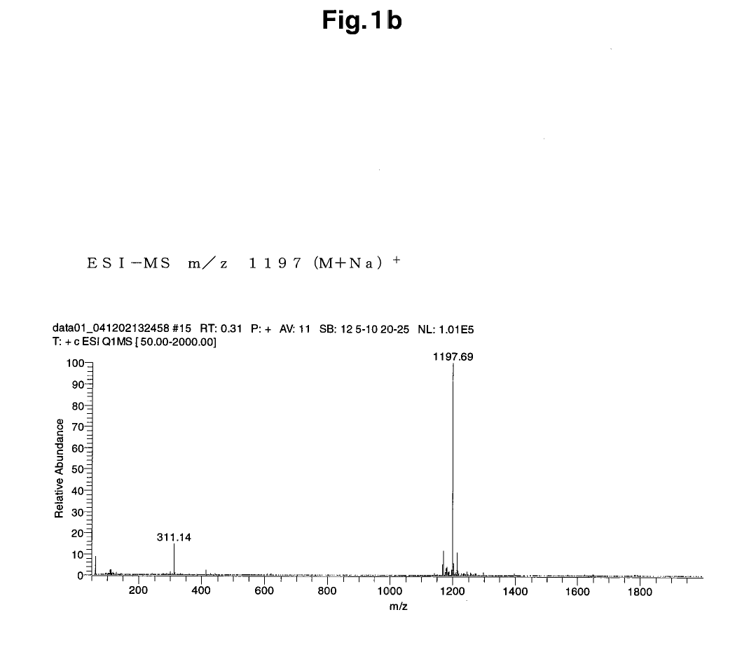

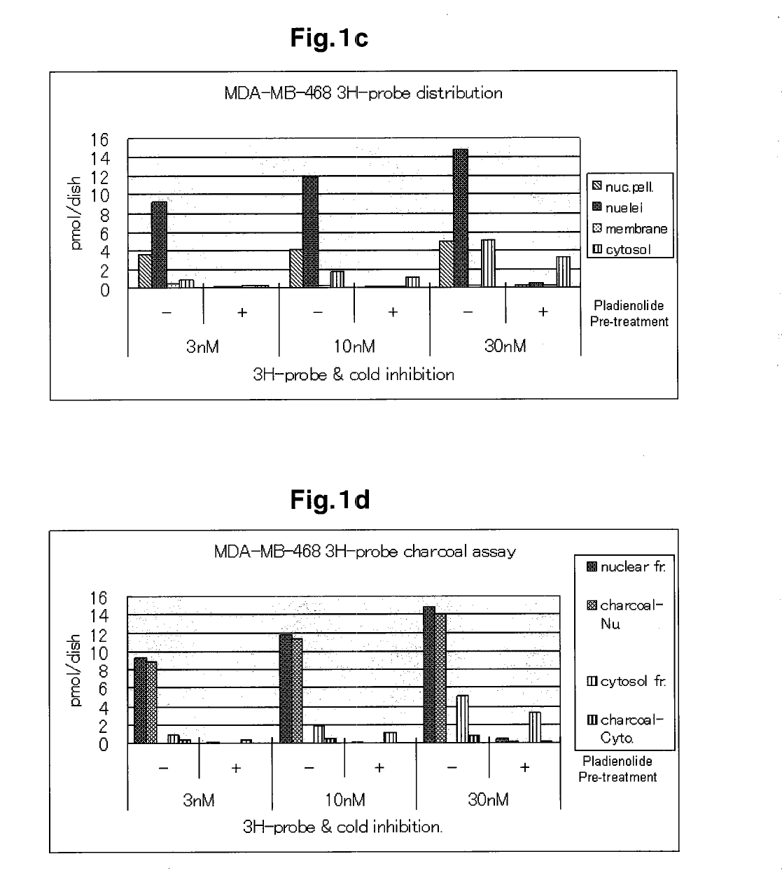

¹H-NMR Spectrum (CDCl₃, 400 MHz): FIG. 1a, (CD-OD, 700 MHz) δ(ppm): 0.83 (3H, m), 0.90 (3H, d, J=7 Hz), 0.93 (3H, t, J=7 Hz), 1.07 (3H, d, J=7 Hz), 1.17 (3H, s), 1.19 (1H, m), 1.23-1.90 (20H, in), 1.71 (3H, s), 2.13-2.57 (6H, in), 2.62-2.74 (3H, m), 2.84-3.35 (10H, m), 3.40-4.10 (6H, m), 4.22-4.52 (2H, m), 4.80-5.10 (3H, m), 5.52 (1H, m), 5.62-5.72 (2H, m), 6.08 (1H, d, J=10.4 Hz), 6.30 (1H, tit), 7.30-7.37 (2H, m), 7.90-8.00 (2H, m)

ESI-MS m/z 1197 (M+Na)⁺: FIG. 1b

Using the synthesized labeled pladienolides, the target protein of pladienolides was identified as described in Examples B1 to B7 below. In addition, it is described in Examples B2, B7, B8 and B9 that the labeled pladienolides can be used to examine that a test compound acts on (binds to) U2 snRNP, preferably SF3b, and more preferably SAP130.

### Example B1

### Intracellular Distribution of Tritium Probe Compound

The tritiated probe compound produced in Example A1 was added to human breast cancer cells MDA-MB-468 (ATCC HTB-132) cultured on a 15-cm dish (>80% confluent), resulting in a concentration of 3 to 30 nM. The obtained mixture was then cultured in an incubator at 37° C. for 1 hour. After removing the medium, the culture was then washed with a Hanks' balanced salt solution (10 ml×2). Then, 3 ml of a 10% glycerol hypotonic buffer (10% glycerol, 20 mM NaCl, 1 mM EDTA, 10 mM Tris-HCl, pH=7.8, protease inhibitor cocktail) was added to the resultant. The obtained mixture was left at rest for a while, and the cells were then recovered using a cell scraper. A cell lysate was prepared using a Dounce homogenizer.

The lysate was centrifuged at 2,000 g at 4° C. for 10 minutes, so as to obtain a supernatant (2000 g-sup.) and a precipitate (2000 g-pellet). 2000 g-sup. was further centrifuged at 100,000 g at 4° C. for 60 minutes, so as to obtain a cytosol fraction as a supernatant, and a membrane fraction as a precipitate. 2000 g-pellet was suspended in 0.5 M NaCl, and the suspension was then left at rest for 30 minutes under cooling on ice, so as to elute a nuclear protein. Thereafter, centrifugation was carried out at 3,000 g at 4° C. for 10 minutes, so as to obtain a nuclear fraction as a supernatant, and a nuclear pellet as a precipitate.

The radioactivity of a tritium probe existing in each fraction was measured by the following method. That is, each of the cytosol fraction and the nuclear fraction was uniformly mixed with Hionic-Fluor (Perkin Elmer). On the other hand, each of the membrane fraction and the nuclear pellet was dissolved in Soluene-350 (Packard) (1 ml), and the obtained solution was then mixed with Hionic-Fluor. The radioactivity of each mixture was measured with a liquid scintillation counter.

At the same time, a radioisotope-non-labeled-pladienolide was used to carry out a cold inhibition experiment. That is, the cells were treated with a pladienolide having a concentration (0.15 to 1.5 μM) that was 50 times higher than the tritium probe treatment concentration, and they were then cultured at 37° C. in an incubator for 1 hour. Thereafter, the culture was treated with a tritium probe, and each fraction was prepared. Then, the radioactivity thereof was measured.

The results are shown in FIG. 1c.

The highest radioactivity of the tritium probe was detected in the nuclear fraction in all the treating concentrations ranging from 3 to 30 nM. The second highest radioactivity was detected in the nuclear pellet. Such radioactivity almost completely disappeared after a pre-treatment with pladienolide B.

Moreover, a charcoal assay was carried out on the prepared cytosol fraction and nuclear fraction, so as to examine that the label count in the test sample was derived from a bound probe binding to a protein and the like, or was derived from a non-bound free form.

That is, 1/10 vol. (volume ratio) of a 10% activated charcoal suspension was added to each of the cytosol fraction and the nuclear fraction, followed by a treatment with a vortex for several seconds. The resultant was centrifuged at 15,000 rpm at 4° C. for 5 minutes (TOMY mini MX-15), and the radioactivity of the supernatant was measured in the same manner as that described above.

The obtained results are shown in FIG. 1d.

The radioactivity in the nuclear fraction did not disappear even after a treatment with activated charcoal. In contrast, almost all the radioactivity of the cytosol fraction disappeared.

From the aforementioned results, it was suggested that a target molecule to which the pladienolide has bound exists in the nuclei.

### Example B2

### Competition Assay of Pladienolide Analogues and Derivatives with Tritium Probes

Various pladienolide analogues or derivatives having antitumor activity were added to human large intestine cancer WiDr cells (ATCC CCL-218) cultured on a 15-cm dish (>80% confluent), resulting in a concentration of 500 nM. The obtained mixture was cultured at 37° C. in an incubator for 1 hour. Subsequently, a tritium probe compound was added to the medium, resulting in a concentration of 10 nM, and the obtained mixture was further cultured at 37° C. in an incubator for 1 hour. A nuclear fraction was prepared from each sample by the same method as that described in Example B1, and the radioactivity of the fraction was then measured.

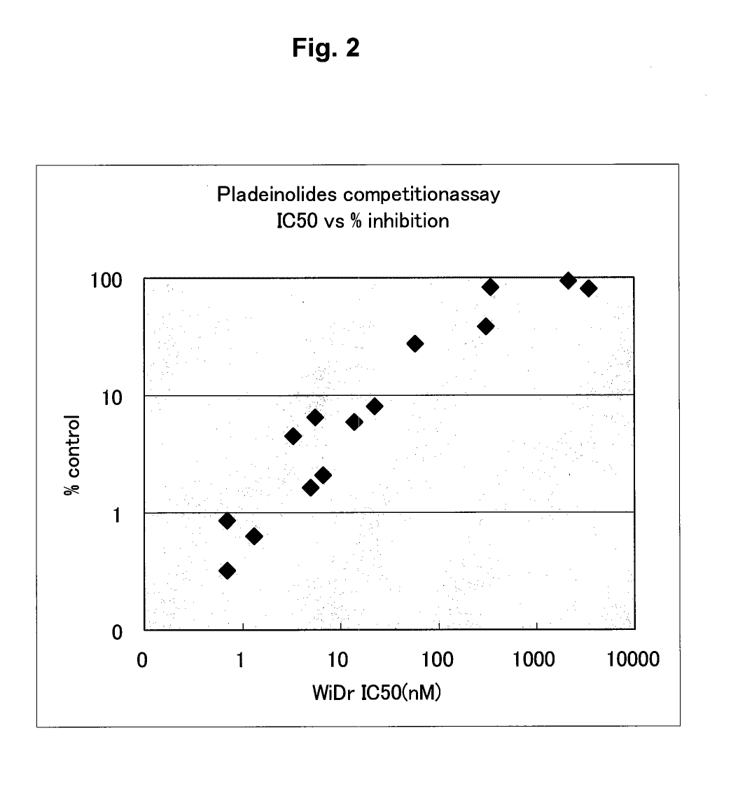

FIG. 2 shows a graph in which each compound was plotted. The longitudinal axis indicates the binding rate (%) of the tritium probe compound that has bound in the presence of various pladienolide analogues or derivatives relative to control samples (no competition), and the horizontal axis indicates IC50 (nM) of the in vitro cytostatic activity of each compound.

The radioactivity of each sample was proportional to IC50 of the in vitro cytostatic activity of the competing pladienolides and the derivatives thereof to the WiDr cells. That is, stronger competitive inhibition was observed in a compound exhibiting strong cytostatic activity.

From such results, it was revealed that a target molecule that was found to exist in the nuclei as a result of examination with a tritium probe was associated with the cytostatic activity of pladienolides.

The pladienolide analogues or derivatives used in the present example are as follows:

(1) the compound of Example A3 of the present application;
(2) the compounds of Examples A-3, A-4, A-5, A-6, A-7, A-8, B-68, B-44, B-50, B36-1 and B36-2 of WO02/060890; and
(3) the compounds of Examples 12 and 45 of WO03/099813.

### Example B3

### Observation of Intracellular Localization of Fluorescent Probe

1 μM of the fluorescent probe produced in Example A2 was added to HeLa cells, and the obtained mixture was incubated at 37° C. in an incubator for 1 hour. Thereafter, the medium was removed, and the remaining culture was further cultured for 1 hour in a fresh medium without probes and non-specifically adsorbed compounds were washed. The medium was removed again. The remaining culture was then washed with PBS. Thereafter, the cells were immobilized in 3.7% formaldehyde in PBS.

The immobilized cells were well washed with PBS, and were then permeabilized with 0.5% or 1% Triton X-100 in PBS. Thereafter, blocking was carried out with FBS or a blocking solution, and a primary antibody (anti-SC-35 monoclonal antibody; Sigma; S4045) was added to the resultant, so as to carry out a reaction. The reaction product was washed with PBS, and blocking was carried out again with FBS or a blocking solution. A fluorescent-labeled secondary antibody (TexasRed-labeled; Jackson) was added to the resultant, so as to carry out a reaction. The labeled sample was rapidly rinsed with distilled water, and it was then encapsulated into a Prolong antifade reagent (Molecular Probes). Localization of the fluorescent probe and the anti-SC-35 antibody was observed under a fluorescence microscope (DeltaVision, Applied Precision, Inc.) or a confocal microscope (LSM510; Carl Zeiss).

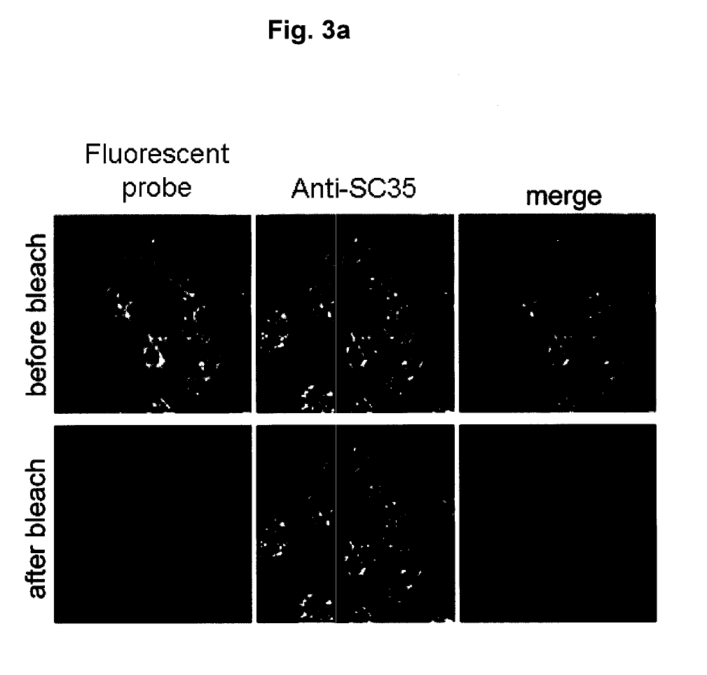

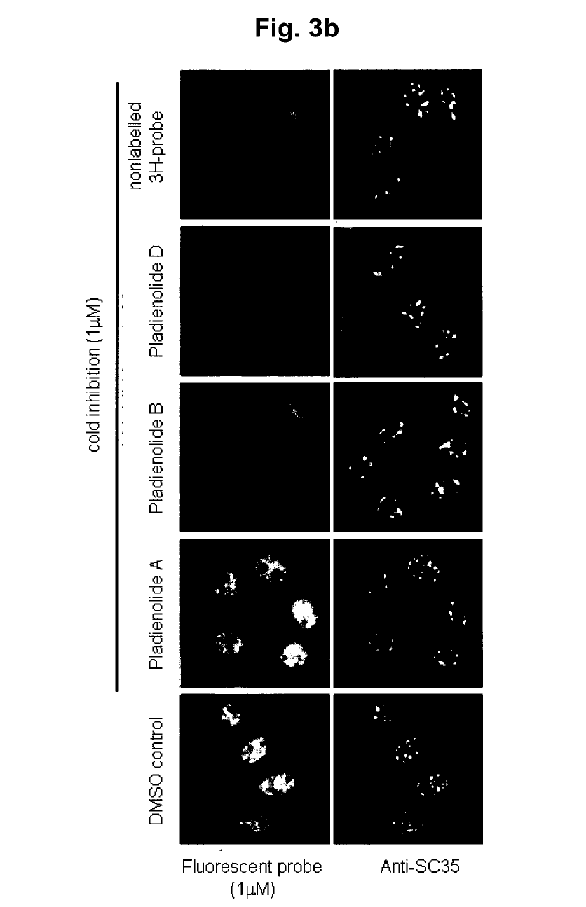

The results are shown in FIG. 3a.

It was confirmed that the fluorescent probe is mainly localized in granular structures in the nuclei (FIG. 3a, upper). These structures corresponded to localization of the anti-SC-35 antibody. SC-35 is a representative molecule of splicing factors and is a marker of nuclear speckles. Thus, it was suggested that the target molecule of pladienolides exists in such nuclear speckles. Moreover, the fluorescence of the fluorescent probe was faded by excessive light irradiation, and the above probe was then photographed under the same above conditions. As a result, no signals were detected in the channel of the fluorescent probe. Thus, it was confirmed that no signals were leaked from anti-SC-35 antibody staining, and that a fluorescent probe-specific signal was detected (FIG. 3a, lower)

Furthermore, pladienolides or the derivatives thereof were used in the aforementioned experimental operations to carry out a competition assay. That is, before addition of the fluorescent probe, 1 μM pladienolides or the derivatives thereof were added to the cells, and the obtained mixture was then cultured for 1 hour. Thereafter, the same above operations for immobilization and staining were performed on the culture, followed by observation with a microscope, so as to confirm localization of the fluorescent probe.

The results are shown in FIG. 3b.

The fluorescence intensity derived from the fluorescent probe contained in each sample was proportional to IC50 of the cytostatic activity of the competing pladienolides or the derivatives thereof (pladienolide A >non-labeled tritium probe compound >pladienolides B and D). That is, stronger competitive inhibition was observed in a compound exhibiting strong cytostatic activity, and the fluorescence derived from the probe almost completely disappeared (FIG. 3b, left column). Moreover, it was observed that the number of nuclear speckles stained with the anti-SC-35 antibody was decreased and that the size thereof was enlarged (FIG. 3b, right column).

From such results, it was revealed that the target molecule confirmed to exist in the nuclear speckles by the experiments using a fluorescent probe, was associated with the cytostatic activity of pladienolides. In addition, a non-labeled tritium probe compound having a structure identical to that of a tritium probe competed with a fluorescent probe at an intensity level that was proportional to the cytostatic activity thereof. Thus, it was revealed that target molecules targeted by these two probes were identical.

### Example B4

### Immunoprecipitation Experiment of Nuclear Fraction Prepared from Tritium Probe-Treated Cells

MDA-MB-468 cells were cultured in the presence of a 30 nM tritium probe for 1 hour. Thereafter, a nuclear fraction was prepared by the same method as that described in Example B1. A dilution buffer was added to the nuclear fraction to a final concentration of 0.15 M NaCl, 0.05% CA-630 and 40 mM tris-HCl (pH=7.5), so as to prepare an immunoprecipitation solution.

To this solution, each of antibodies reacting with splicing factors, transcriptional factors, or molecules associated with such factors were added to a concentration of 1 μg/ml. The obtained mixture was slowly stirred for approximately 12 hours in a low-temperature chamber of 4° C., using a sample rotator. Thereafter, 20 μl of 50% Protein A/G-agarose conjugate suspension was added to the reaction solution, and the obtained mixture was further stirred for 2 hours in the low-temperature chamber.

The agarose resin was recovered, and it was then washed with a washing solution (0.15 M NaCl, 0.05% CA-630, 20 mM tris-HCl, pH=7.5). An SDS sample buffer was added to the resultant solution, and the obtained mixture was then stirred with a vortex. The reaction solution was boiled at 99° C. for 10 minutes, so as to elute the immunoprecipitated protein. The eluant was mixed homogeneously with Hionic-Fluor, and the radioactivity of a tritium probe existing in the eluant was then measured with a liquid scintillation counter.

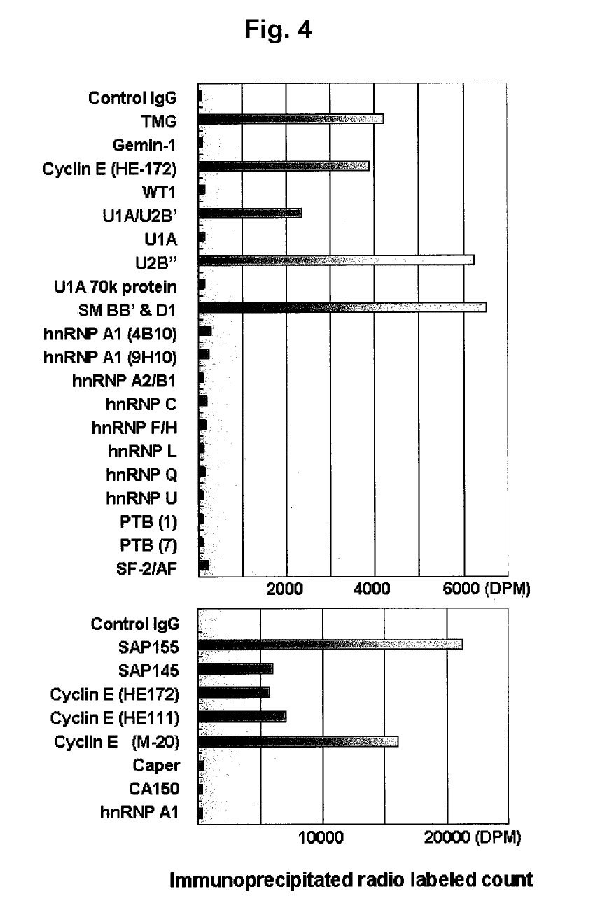

The results are shown in FIG. 4.

The experiment was carried out using approximately 40 types of antibodies. As a result, specific radioactivity was observed in a TMG antibody (Oncogene NA02), a U1A/U2B″ antibody (Progen 57035), a U2B″ antibody (Progen 57036), an SM BB′ & D1 antibody (Progen 57032), an SAP155 antibody (MBL D221-3), an SAP145 antibody (Santa Cruiz sc-14279), and a cyclin E antibody (Zymed 32-1500, Santa Cruiz sc-248, and Santa Cruiz sc-481). All of these antibodies are antibodies reacting with constitutional proteins of U2 snRNP, or a protein that reportedly forms a complex with U2 snRNP (cyclin E).

From such results, it was revealed that the target molecule of pladienolide is a protein existing in the U2 snRNP complex.

### Example B5

### Detection of Bound Protein by Treatment with Photoaffinity Biotin Probe

2000 g-pellet was prepared from MDA-MB-468 cells by the method described in Example B1. This pellet was suspended in a CSK buffer (300 mM sucrose, 100 mM NaCl, 3 mM MgCl₂, 10 mM PIPES, pH=7.0, protease inhibitor cocktail), and the obtained suspension was then treated with the 1.5 μM photoaffinity biotin probe synthesized in Example A3. The suspension was left at rest at 4° C. for 1 hour, and it was then centrifuged at 2000 g at 4° C. for 10 minutes. The supernatant was removed, and a nuclear fraction was then prepared from the obtained precipitate by the same method as that described in Example B1. Thereafter, an immunoprecipitation solution was prepared by the sable method as that described in Example B4. Using antibodies regarding which immunoprecipitation of the target molecules had been proved, an immunoprecipitation experiment was carried out.

UV (365 nm and 302 nm) was applied to the obtained immunoprecipitate (agarose resin), and a crosslinking reaction was carried out. An SDS scruple buffer was added to the reaction product, and the obtained mixture was then stirred with a vortex, followed by boiling at 99° C. for 10 minutes, so as to elute the immunoprecipitated proteins. The eluant was subjected to SDS-PAGE and blotting onto a PVDF membrane in accordance with common methods. Thereafter, the resultant was treated with streptavidin-HRP, so as to detect probe-bound proteins.

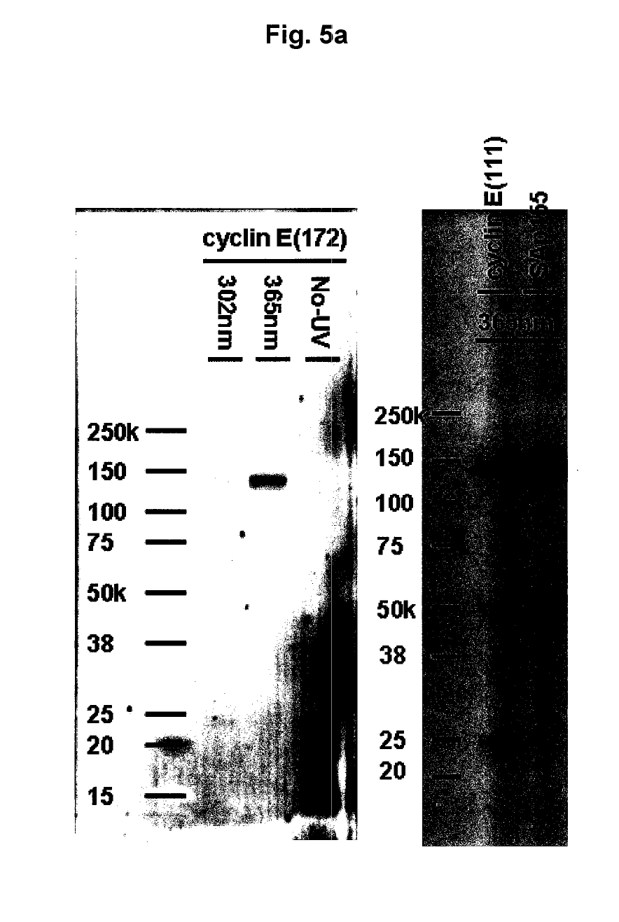

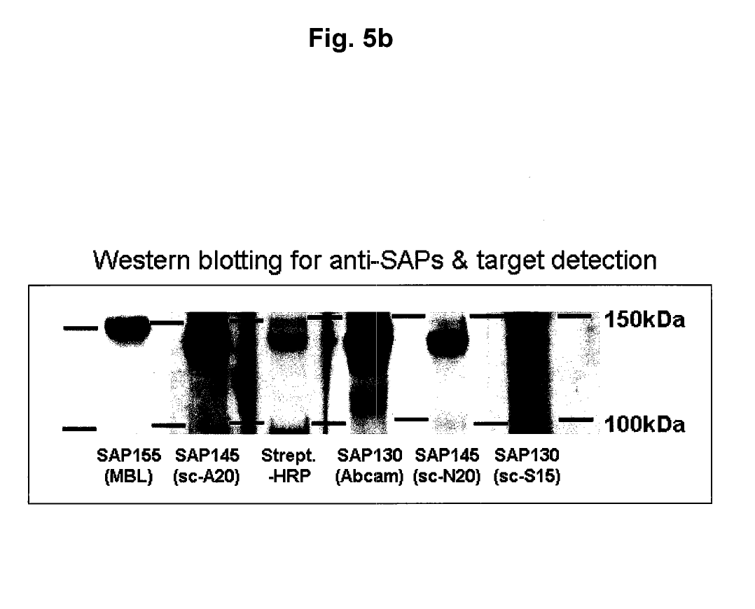

The results are shown in FIG. 5a.

In all antibodies, a single band was detected at 130-140 kDa.

Subsequently, the same sample (blotting membrane) was subjected to Western blotting using an SAP155 antibody, an SAP145 antibody, and SAP 130 antibody. The band position of each of such proteins was compared with the band position of the target molecule, to which a photoaffinity probe had bound.

The results are shown in FIG. 5b.

The position of the target molecule clearly differed from the position of SAP 155. On the other hand, the target molecule, SAP145, and SAP130 were observed in almost the same position.

From such results, it was suggested that the target molecule was SAP145 or SAP130.

### Example B6

### Acquisition of GFP-145 Strain and GFP-130 Strain

### (1) Construction of Plasmid

In order to acquire a strain stably expressing a GFP-conjugated SAP130 protein, a pEGFP-SAP130 plasmid was produced. The pEGFP-SAP130 was produced by inserting, into the NheI-PmeI site of a pcDNA3.1(−) vector (Invitrogen), an approximately 4.4 kbp DNA fragment as shown in SEQ ID NO: 1 (an NheI-PmeI fragment of GFP-SAP130; 4,432 bp), which was constituted with an EGFP protein coding sequence (derived from pEGFP-N2; CLONTECH), a human SAP130 (SF3B3) protein coding sequence (GenBank Accession#: NM_{—}012426), and a linker sequence that connects such sequences to one another. It was anticipated that a GFP-SAP130 protein having the amino acid sequence as shown in SEQ ID NO: 2 was allowed to express in cells, into which the above plasmid had been introduced.

In order to acquire a strain stably expressing a GFP-conjugated SAP145 protein, a pEF1-GFP-SAP145 plasmid was produced. The pEF1-GFP-SAP145 was produced by inserting, into the HindIII-PmeI site of a pEF1-Myc/His A vector (Invitrogen), an approximately 3.5 kbp DNA fragment as shown in SEQ ID NO: 3 (a HindIII-PmeI fragment of GFP-SAP145; 3,454 bp), which was constituted with an EGFP protein coding sequence (derived from pEGFP-N2; CLONTECH), a human SAP145 (SF3B2) protein coding sequence (GenBank Accession#: NM_{—}006842), and a linker sequence that connects such sequences to one another. It was anticipated that a GFP-SAP145 protein having the amino acid sequence as shown in SEQ ID NO: 4 was allowed to express in cells, into which the above plasmid had been introduced.

### (2) Establishment of Stably Expressing Strain

Each of the pEF1-GFP-SAP145 and pEGFP-SAP130 was introduced into HeLa cells, using Lipofectamine 2000 reagent (Invitrogen). Forty hours after introduction of the gene, selection of gene-introduced cells was initiated by addition of Geneticin (Invitrogen) with a final concentration of 500 μg/ml. Fourteen days after the gene introduction, clones emitting GFP fluorescence were marked by fluorescence microscope observation, and were then picked up. Thereafter, a single colony was isolated by diluted passage. Clone #2 (GFP-145 strain) derived from pEF1-GFP-SAP145, and Clone #3 (GFP-130 strain) derived from pEGFP-SAP130, were used in the subsequent experiments.

### Example B7

### Detection Experiment of Band Shift Using GFP-Fused SAP145 or GFP-Fused SAP130 Proteins

SAP145 and SAP130, which were considered to be target molecules, were detected in almost the same position on SDS-PAGE. Thus, strains stably expressing a GFP-fused SAP145 or GFP-fused SAP 130 protein were prepared, and an experiment was then Buried out using a photoaffinity biotin probe.

A 1.5 μM photoaffinity biotin probe was added to HeLa cells stably expressing GFP-SAP145, and the obtained mixture was then cultured for 1 hour. After washing the cells with PBS, M-PER (PIERCE) was then added thereto, followed by stirring at 4° C. for 1 hour. Thereafter, 1/10 vol. of 1.5 M NaCl-0.5% Tween 20 solution was added to the reaction solution, and the obtained mixture was then treated with a vortex (10 sec.×3). The resultant was centrifuged at 15,000 rpm at 4° C. for 5 minutes (TOMY MX-15). The thus obtained supernatant was filtrated with a 0.45-μm membrane, and the filtrate was used in the following immunoprecipitation experiment.

A GFP antibody (Q-BIOgene AFP5002) or an SAP155 antibody (MBL D221-3) (10 μg/ml) was added to the immunoprecipitation solution, and the obtained mixture was then stirred slowly with a rotator at 4° C. for 30 minutes. Subsequently, 1/10 vol. of protein A/G-agarose (50% suspension) was added to the immunoprecipitation solution, and the obtained mixture was further stirred at 4° C. for 1 hour in the same manner as above. The agarose resin was washed with a washing buffer 3 times, and UV was then applied to the resultant resin by the same method as that described in Example B5. Thereafter, the protein bound to the resin was eluted, and it was then subjected to SDS-PAGE and blotting. The resultant was treated with streptavidin-HRP, so as to detect a protein to which the probe had bound.

Moreover, using the same samples. SAP145 and SAP130 were detected with the SAP145 antibody and the SAP130 antibody, respectively.

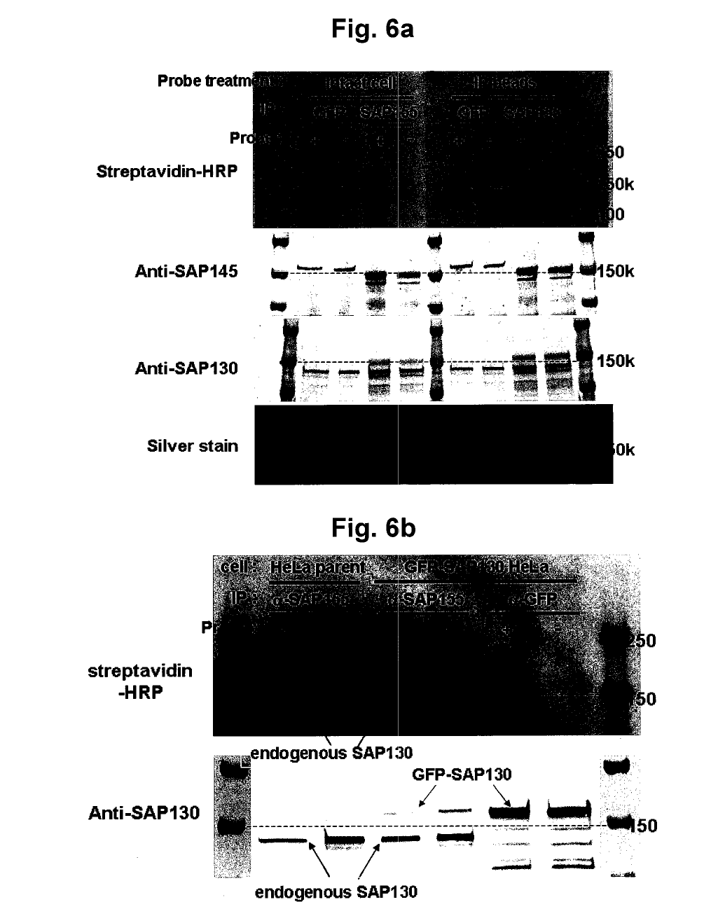

The results are shown in FIG. 6a (probe treatment: intact cell).

In both samples immunoprecipitated with the GFP antibody and the SAP155 antibody, protein bands to which the probes had bound were detected in positions ranging from 130 to 145 kDa. These bands were not detected in a probe-non-treatment sample. Accordingly, it was determined that these were signals derived from the probes binding to target molecules.

The same samples were subjected to Western blotting using the SAP145 antibody and the SAP130 antibody. In the sample immunoprecipitated with the GFP antibody, no endogenous SAP145 was detected in a position ranging from 130 to 145 kDa. It was revealed that only the SAP145-GFP fusion protein was present in a shifted position of approximately 170 kDa. On the other hand, from the experimental results using the SAP130 antibody, it was revealed that SAP130 was present in a position wherein the probe had been detected.

As a result, it was revealed that SAP145 was not present in the band position of the target molecule, and that only SAP130 was present. Thus, it was strongly suggested that the target molecule was SAP130.

Furthermore, an agarose resin prepared from probe-untreated cells was treated with a probe in the aforementioned experimental operations. Thereafter, UV irradiation and elution of a protein were carried out by the same operations as above, and it was examined whether or not the same band was detected in the obtained sample.

The results are shown in FIG. 6a (probe treatment: IP beads).

Even in a case where the previously immunoprecipitated sample was treated with the probe, the same results as those in the case of the treatment of intact cells were obtained. Accordingly, it was revealed that pladienolide has affinity even for a complex comprising the immunoprecipitated target molecule.

Subsequently, the same experiment was carried out using HeLa cells stably expressing GFP-SAP130.

The results are shown in FIG. 6b.

In a sample immunoprecipitated with the SAP155 antibody, two bands, namely, a band shifted to approximately 170 kDa and a band in the conventional position ranging from 130 to 145 kDa, were detected. On the other hand, in a sample immunoprecipitated with the GFP antibody, only a band shifted to 170 kDa was detected. These bands were not detected in a probe-non-treatment sample. Accordingly, it is determined that these were signals derived from the probes binding to target molecules.

The same samples were subjected to Western blotting using the SAP130 antibody. In a sample immunoprecipitated with the SAP155 antibody, signals were detected in a position wherein a band shift had been observed and in the conventional SAP130 position. On the other hand, in a sample immunoprecipitated with the GFP antibody, an SAP130 signal was observed only in the position wherein a band shift had been observed.

From such results, it was determined that a protein to which a photoaffinity biotin probe has bound is not SAP145 but SAP130.

### Example B8

### Competition Assay of Pladieno Analogues and Derivatives with Photoaffinity Biotin Probes

Probe-untreated HeLa cells stably expressing GFP-SAP145 were subjected to an immunoprecipitation experiment by the same method as that described in Example B7 using a GFP antibody and an SAP155 antibody, so as to prepare an immunoprecipitate sample (agarose resin). This resin was suspended in a washing buffer, and was pre-treated with pladienolides and the derivative thereof (E7107) at 75 μM, followed by stirring with a rotator at 4° C. for 30 minutes. Subsequently, it was treated with a photoaffinity biotin probe at 1.5 μM, and it was further stirred for 30 minutes in the same manner as above. The resin was washed with a washing buffer. Thereafter, UV irradiation and elution of a protein were carried out by the same method as described in Example B7. The obtained sample was subjected to SDS-PAGE and blotting to examine whether or not the band derived from the probe disappeared, by the competition between the photoaffinity biotin probe and the pre-treating pladienolide compound.

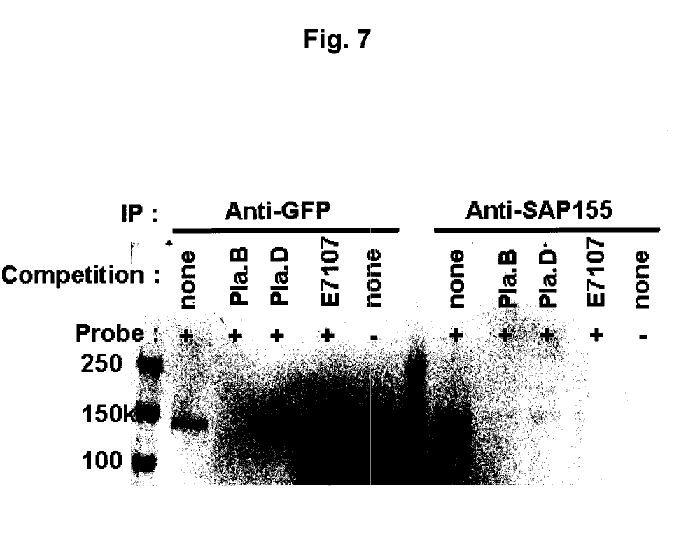

The results are shown in FIG. 7.

In both cases of the GFP antibody sample and the SAP155 antibody sample, the band derived from the probe disappeared by competition with the pladienolide compounds (Pladienolide B, D and E7107). Accordingly, it can be said that the protein bounded by the photoaffinity biotin probe is the pladienolide-bound protein.

As stated above, a target molecule of pladienolide was pursued using several probe compounds. As a result, it was concluded that such a target molecule of pladienolide is SAP130, which is one of SF3b constituent proteins.

### Example B9

For the purpose of efficiently and quantitatively selecting compounds that bind to SF3b out of many evaluation samples, a simple evaluation method using High content screening, machine and fluorescent probes has been established.

90 μl (1.0×10⁴ cells/well) of HeLa cells were inoculated on a 96-well plate used for fluorescence measurement, and they were then cultured overnight at 37° C. in a 5% CO₂ incubator. A dilution series of E7107, which had been found to bind to SF3b from the results of Example B8, was prepared, and it was then added to the cultured HeLa cells to the following final concentrations (0, 0.15, 0.46, 1.4, 4.1, 12, 37, 111, 333, and 1000 nM). Thereafter, the mixture was cultured at 37° C. in a 5% CO₂ incubator for 30 minutes. Subsequently, 50 μl of fluorescent probes that had been adjusted to 3 μM was added to the culture (final concentration: 1 μM), and the mixture was then reacted for 2 hours at 37° C. in a 5% CO₂ incubator. The medium was removed, and the resultant was fixed with 2% paraformaldehyde for 5 minutes. The resultant was washed with PBS, was then treated with 0.1% Triton X-100/PBS for 5 minutes, and was then washed with PBS. Thereafter, nuclei were stained with 1 μg/ml H33342 (SIGMA), and the image of such nuclear staining and fluorescent probes was obtained using INCell analyzer (GE Healthcare). The obtained image was subjected to image analysis software, Developer (GE Healthcare), so as to analyze the fluorescence intensity of the fluorescent probes in the nuclei and examine whether or not accumulation of the fluorescent probes into the nuclei is inhibited by E7107 binding to SF3b.

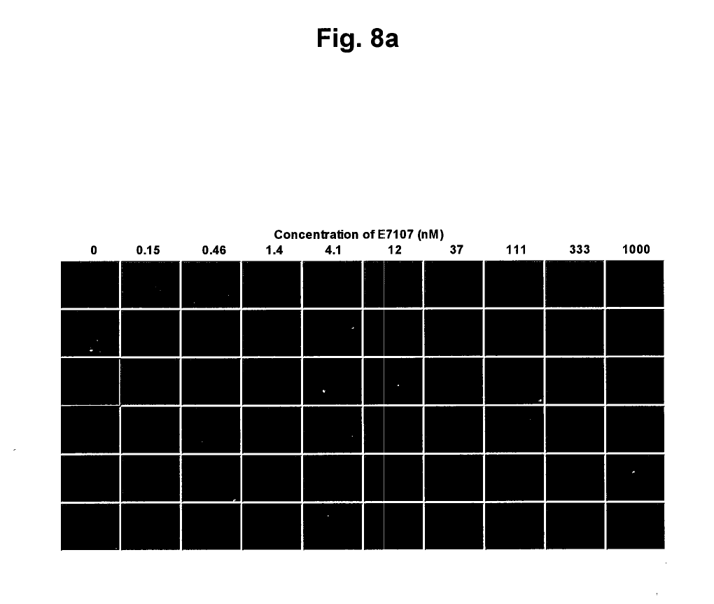

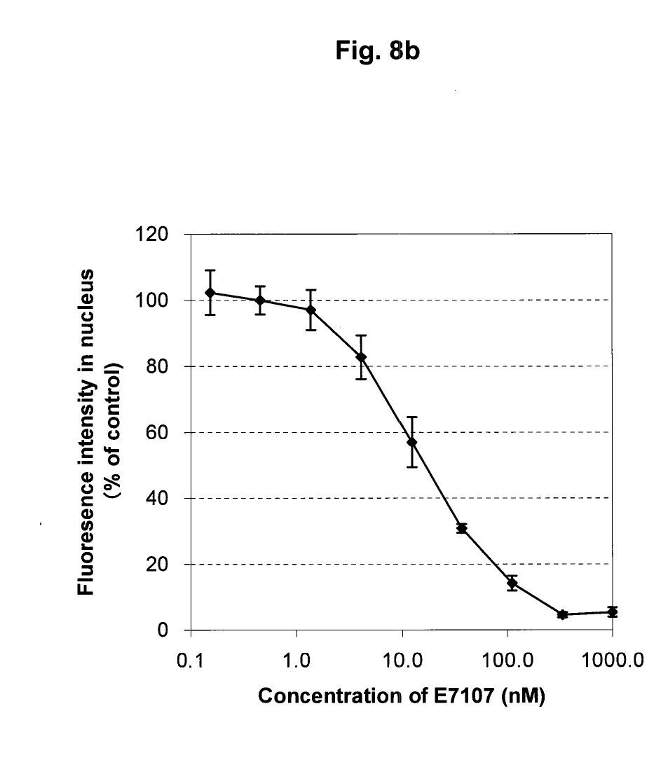

A representative example (3 views were photographed for each well) of the images of the fluorescent probes used in the image analyses (6 images in the longitudinal direction had the same concentration) is shown in FIG. 8a, and the summary of such image analyses is shown in FIG. 8b. E7107 concentration-dependently inhibited accumulation of the fluorescent probes into the nuclei, and thus this method enabled a simple, quantitative measurement of the compound binding to SF3b.

1. A method of measuring the binding activity of a test compound to a splicing factor 3b (SF3b), which comprises the following steps of:
(a) contacting a labeled compound represented by the following formula (I), and a test compound with a cell or a cell fraction; and
(b) measuring the distribution of the bound labeled compound, 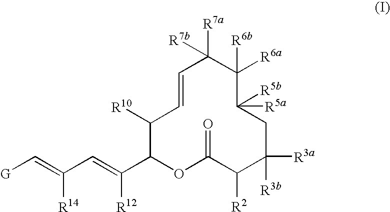
wherein R², R¹⁰, R¹², and R¹⁴ are independently selected from the group consisting of hydrogen and methyl;
R^{3a}, R^{3b}, R^{5a}, R^{5b}, R^{6a}, and R^{6b} are independently selected from the group consisting of:
(1) hydrogen,
(2) hydroxy,
(3) any one of the following groups, each of which may have substituent(s),
(a) C₁₋₂₂ alkyl,
(b) C₁₋₂₂ alkoxy,
(c) ArCH₂O— (wherein Ar represents C₆₋₁₄ aryl or 5- to 14-membered ring heteroaryl, each of which may have substituent(s)),
(d) C₂₋₂₂ acyloxy,
(e) C₃₋₂₂ unsaturated acyloxy,
(f) —OCOR^{CO} (wherein R^{CO} represents
(i) C₆₋₁₄ aryl,
(ii) 5- to 14-membered ring heteroaryl,
(iii) C₁₋₂₂ alkoxy,
(iv) unsaturated C₂₋₂₂ alkoxy,
(v) C₆₋₁₄ aryloxy, or
(vi) 5- to 14-membered ring heteroaryloxy, each of which may have substituent(s)),
(g) C₁₋₂₂ alkylsulfonyloxy,
(h) benzenesulfonyloxy, and
(i) —OSiR^{S1}R^{S2}R^{S3} (wherein R^{s1}, R^{s2}, and R^{s3} are the same as or different from one another and each represents methyl, ethyl, i-propyl, t-butyl, or phenyl),
(4) halogen, or
(5) —R^{M}—NR^{N1}R^{N2} {wherein R^{M} represents a single bond or —O—CO—;
and R^{N1} and R^{N2} are as follows,
1) R^{N1} and R^{N2} are the same as or different from each other and each represents (a) hydrogen or
(b) any one of the following groups, each of which may have substituent(s):
(i) C₁₋₂₂ alkyl,
(ii) unsaturated C₃₋₂₂ alkyl,
(iii) C₂₋₂₂ acyl,
(iv) unsaturated C₃₋₂₂ acyl,
(v) C₆₋₁₄ aryl,
(vi) 5- to 14-membered ring heteroaryl,
(vii) benzyl,
(viii) C₁₋₂₂ alkylsulfonyl or
(ix) benzenesulfonyl, or
2) NR^{N1}R^{N2} may together represent 3- to 14-membered ring nitrogen-containing nonaromatic hetero ring, which may have substituent(s)};
R^{7a} and R^{7b} are as follows,
(1) R^{7a} and R^{7b} are the same as or different from each other and each represents,
1) hydrogen,
2) —OR^{H} (wherein R^{H} represents hydrogen, methyl, or acetyl),
3) —OR^{D} {wherein R^{D} represents any one of the following groups, each of which may have substituent(s),
(i) C₁₋₂₂ alkyl (in the case of methyl, it must have substituent(s)),
(ii) —CH₂Ar,
(iii) C₃₋₂₂ acyl,
(iv) C₃₋₂₂ unsaturated acyl,
(v) —COR^{CO},
(vi) C₁₋₂₂ alkylsulfonyl,
(vii) benzenesulfonyl or
(viii) —SiR^{S1}R^{S2}R^{S3}}, or
4) —R^{M}—NR^{N1}R^{N2}, or
(2) R^{7a} and R^{7b} together represent
1) a ketone structure (═O) or
2) an oxime structure (═NOR^{OX}, wherein R^{OX} represents
(a) C₁₋₂₂ alkyl,
(b) unsaturated C₃₋₂₂ alkyl,
(c) C₆₋₁₄ aryl,
(d) 5- to 14-membered ring heteroaryl, or
(e) benzyl, each of which may have substituent(s));
further,
R^{3a} and R^{3b} may together represent a ketone structure (═O) or an oxime structure (═NOR^{OX});
still further,
R^{6a} and R^{6b} may together represent a spiro-oxirane ring or exomethylene; and
still further,
either R^{6a} or R^{6b} and either R^{7a} or R^{7b} may together form a 1,3-dioxolane ring;
G represents, 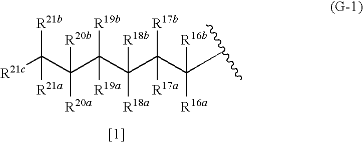
wherein R^{16a} and R^{16b} are independently selected from the group consisting of hydrogen, methyl, or hydroxyl;
R^{17a}R^{17b}, R^{18a}, R^{18b}, R^{19a}, R^{19b}, R^{20a}, R^{20b}, R^{21a}, and R^{21b} are independently selected from the group consisting of:
(1) hydrogen,
(2) methyl, which may have substituent(s),
(1) —OR^{H},
(2) —OR^{D},
(3) halogen, and
(4) —R^{M}—NR^{N1}R^{N2};
R^{21c} represents (1) hydrogen or (2) 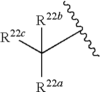
(wherein R^{22a}, R^{22b}, and R^{22c} are independently selected from the group consisting of (a) hydrogen, (b) methyl, (c) hydroxy, (d) —OR^{H}, (e) —OR^{D}, (f) —R^{M}—NR^{N1}R^{N2}, and (g) halogen; further,
either R^{18a} or R^{18b} and either R^{19a} or R^{19b} may together form a single bond, so as to represent a partial structure 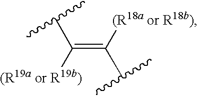
or they may bind to oxygen, so as to represent a partial
structure 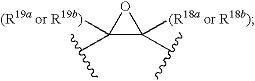
either R^{19a} or R^{19b} and either R^{20a} or R^{20b} may together form a single bond, so as to represent 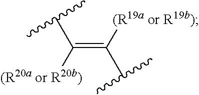
R^{21a} and R^{21b} may together represent (a) a ketone structure (═O) or (b) an oxime structure (═NOR^{OX});
either R^{21a} or R^{21b} and either R^{22a} or R^{22b} may together represent a partial structure 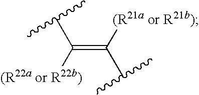
either R^{19a} or R^{19b} and either R^{21a} or R^{21b} may together represent a partial structure 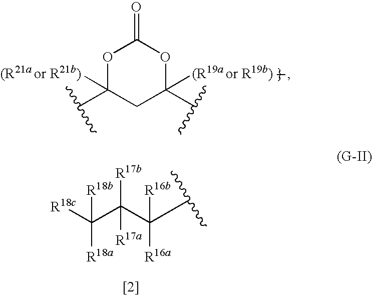
and R¹⁸c represents (1) hydrogen or (2) the formula: 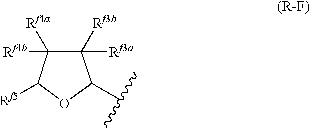
wherein R^{f3a}, R^{f3b}, R^{f4a}, and R^{f4b} are independently selected from the group consisting of: hydrogen, methyl, hydroxy, methoxy, and acetoxy; and R^{f5} represents methyl or ethyl or 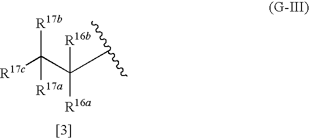
wherein R^{16a} R^{17c} represents (1) hydrogen or (2) the formula: 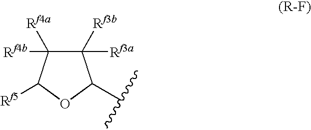
wherein R^{f3a}, R^{f3b}, R^{f4a}, and R^{f4b} are independently selected from the group consisting of: hydrogen, methyl, hydroxy, methoxy, and acetoxy; and
R^{f5} represents methyl or ethyl.

2. The method according to claim 1, wherein the compound represented by the formula (I) is a compound represented by the following formula (IV): 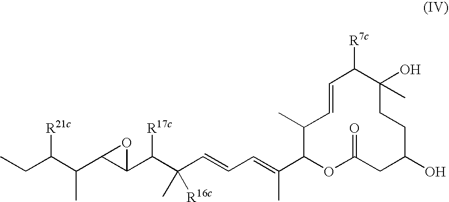
wherein R^{16c}, R^{17c}, and R^{21c} are independently selected from the group consisting of hydrogen, hydroxy, and methoxymethyl; and
R^{7c} represents hydroxy, acetoxy, or O—CO—NR^{N1′}R^{N2′}; wherein R^{N1′} and R^{N2′} are independently selected from the group consisting of hydrogen and C₁₋₆ alkyl.

3. The method according to claim 1, wherein the compound represented by the formula (I) is a compound represented by the following formula (V): 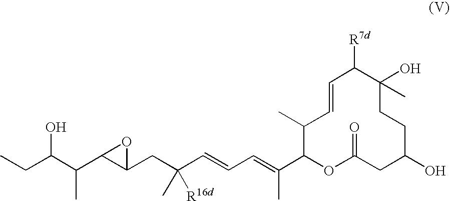
wherein R^{16d} represents hydrogen or hydroxy; and
R^{7d} represents hydroxy, acetoxy, or O—CO—NHR^{N1′} and wherein R^{N1′} represents C₁₋₆ alkyl.

4. The method according to claim 1, wherein the activity of the test compound to bind to SAP130 in SF3b is measured.

5. The method according to claim 1, wherein the label is a radioisotope label.

6. The method according to claim 1, wherein the label is a fluorescent label.

7. The method according to claim 1, wherein the label is a biotin label.

8. The method according to claim 1, wherein the label comprises a compound that binds to a protein as a result of exposure to light.

9. The method according to claim 1, wherein, in the step (b), the amount of the labeled compound that is distributed in nuclei is measured, so as to measure the binding activity of the test compound to SF3b.

10. The method according to claim 1, wherein, in the step (b), the amount of the labeled compound that is distributed in nuclear speckles is measured, so as to measure the binding activity of the test compound to SF3b.

11. The method according to claim 1, wherein, in the step (b), the pattern of distribution of the labeled compound in nuclear speckles is measured, so as to measure the binding activity of the test compound to SF3b.

12. The method according to claim 1, wherein, in the step (b), the amount of the labeled compound that is distributed in SF3b-bound state is measured, so as to measure the binding activity of the test compound to SF3b.

13. The method according to claim 1, wherein, in the step (b), the amount of the labeled compound that is distributed in SAP130-bound state is measured, so as to measure the binding activity of the test compound to SF3b.

14. An anticancer agent binding to SF3b determined to have activity to bind to SF3b by the method according to claim 1.

15. An anticancer agent binding to SAP130 determined to have activity to bind to SAP130 by the method according to claim 1.

16. A labeled compound that is any one of those represented by the following formulas (II): [chemical structure]
